(19) United States
(12) Patent Application Publication
(10) Pub. No.: US 2008/0274171 A1
(43) Pub. Date: Nov. 6, 2008

(54) Renin Inhibitors Nitroderivatives

(76) Inventors: Nicoletta Almirante, Milano (IT); Angela Monopoli, Milano (IT); Ennio Ongini, Segrate(Milano) (IT)

Correspondence Address: ARENT FOX LLP, 1050 CONNECTICUT AVENUE, N.W., SUITE 400, WASHINGTON, DC, 20036, US

(21) Appl. No.: 12/090,640

(22) PCT Filed: Oct. 2, 2006
(86) PCT No.: PCT/EP2006/066952

§ 371 Date: Apr. 17, 2008

Related U.S. Application Data
(60) Provisional application No. 60/727,550, filed on Oct. 18, 2005.

(51) Int. Cl. A61K 9/127; C07C 69/34; A61K 9/28; C07K 5/087; A61P 9/00; A61K 9/48; A61K 31/222

(52) U.S. Cl. 424/450; 560/193; 514/551; 424/474; 424/463; 514/18; 530/331

(57) ABSTRACT

Renin inhibitors nitroderivatives of formula (I):

A-(X₀—ONO₂)ₛ (I)

having wider pharmacological activity and enhanced tolerability. They can be employed for treating or preventing congestive heart failure, coronary diseases, cardiac insufficiency, left ventricular dysfunction and hypertrophy, cardiac fibrosis, myocardial ischemia, stroke, atherosclerosis, restenosis post angioplasty, renal insufficiency, renal ischemia, renal failure, renal fibrosis, glomerulonephritis, renal colic, ocular and pulmonary hypertension, glaucoma, hypertension, diabetic complications such as nephropathy, vasculopathy and neuropathy, peripheral vascular diseases, liver fibrosis, portal hypertension, metabolic syndrome, erectile dysfunction, complications after vascular or cardiac surgery, complications of treatment with immunosuppressive agents after organ transplantation, hyperaldosteronism, lung fibrosis, scleroderma, anxiety, cognitive disorders.

The present invention relates to nitroderivatives of renin inhibitors, pharmaceutical compositions containing them and their use for the treatment or prophylaxis of cardiovascular, renal and chronic liver diseases, inflammatory processes and metabolic syndrome.

Renin is a proteolytic enzyme which is predominantly released into the blood from the kidney. It cleaves its natural substrate, angiotensinogen, releasing decapeptide, angiotensin I. This is in turn cleaved by converting enzyme (ACE) in the lung, kidney and other tissues to the octapeptide angiotensin II, which has an effect on blood pressure. Angiotensin II raises blood pressure both directly by causing arteriolar constriction and indirectly by stimulating release of the sodium-retaining hormone aldosterone from the adrenal gland causing a rise in extracellular fluid volume.

The activity of the renin-angiotensin system can be manipulated pharmacologically by the inhibition of the activity of renin (renin inhibitors), or by the inhibition of the angiotensin converting enzyme (ACE inhibitors) or by blockade of angiotensin II receptors (angiotensin II receptor blockers).

Renin inhibitors have been thought as agents for control of hypertension, congestive heart failure, and hyperaldosteronism. Inefficient absorption, high first-pass metabolism and biliary excretion have constituted an obstacle to the clinical development of this group of drugs.

WO 01/35961 describes methods of treating and/or preventing vascular diseases where nitric oxide insufficiency is a contributing factor by administering a therapeutically effective amount of at least one antioxidant, or a pharmaceutically acceptable salt thereof, and at least one of isosorbide dinitrate and isosorbide mononitrate, and, optionally, at least one nitrosated angiotensin-converting enzyme inhibitor, nitrosated beta-adrenegic blocker, nitrosated calcium channel blocker, nitrosated endothelin antagonist, nitrosated angiotensin II receptor antagonist, nitrosated renin inhibitor, and/or at least one compound used to treat cardiovascular diseases.

WO 2005/023182 describes novel nitrosated and/or nitrosylated cardiovascular compounds or pharmaceutically acceptable salts thereof, and novel compositions comprising at least one nitrosated and/or nitrosylated cardiovascular compound, and, optionally, at least one nitric oxide donor and/or at least one therapeutic agent. The nitrosated and/or nitrosylated cardiovascular compounds are selected from: aldosterone antagonists, angiotensin II antagonists, calcium channel blockers, nitrosated and/or nitrosylated endothelin antagonists, hydralazine compounds, neutral endopeptidase inhibitors and renin inhibitors.

The need was felt to have available new renin inhibitors able not only to eliminate or at least reduce the drawbacks of their parent compounds, but also having an improved pharmacological activity. It has been so surprisingly found that renin inhibitors nitroderivatives of the present invention have a significantly improved overall profile as compared to native compounds both in term of wider pharmacological activity and enhanced tolerability.

In particular, it has been recognized that the renin inhibitors nitroderivatives of the present invention exhibit a strong anti-inflammatory, antithrombotic and antiplatelet activity and can be furthermore employed for treating or preventing congestive heart failure, coronary diseases, cardiac insufficiency, left ventricular dysfunction and hypertrophy, cardiac fibrosis, myocardial ischemia, stroke, atherosclerosis, restenosis post angioplasty, renal insufficiency, renal ischemia, renal failure, renal fibrosis, glomerulonephritis, renal colic, ocular and pulmonary hypertension, glaucoma, hypertension, diabetic complications such as nephropathy, vasculopathy and neuropathy, peripheral vascular diseases, liver fibrosis, portal hypertension, metabolic syndromes, erectile dysfunction, complications after vascular or cardiac surgery, complications of treatment with immunosuppressive agents after organ transplantation, hyperaldosteronism, lung fibrosis, scleroderma, anxiety, cognitive disorders.

Object of the present invention are, therefore, renin inhibitors nitroderivatives of general formula (I) and pharmaceutically acceptable salts or stereoisomers thereof:

A-(X₀—ONO₂)₃ (I)

wherein:
s is an integer equal to 1 or 2;
A is selected from the following groups:

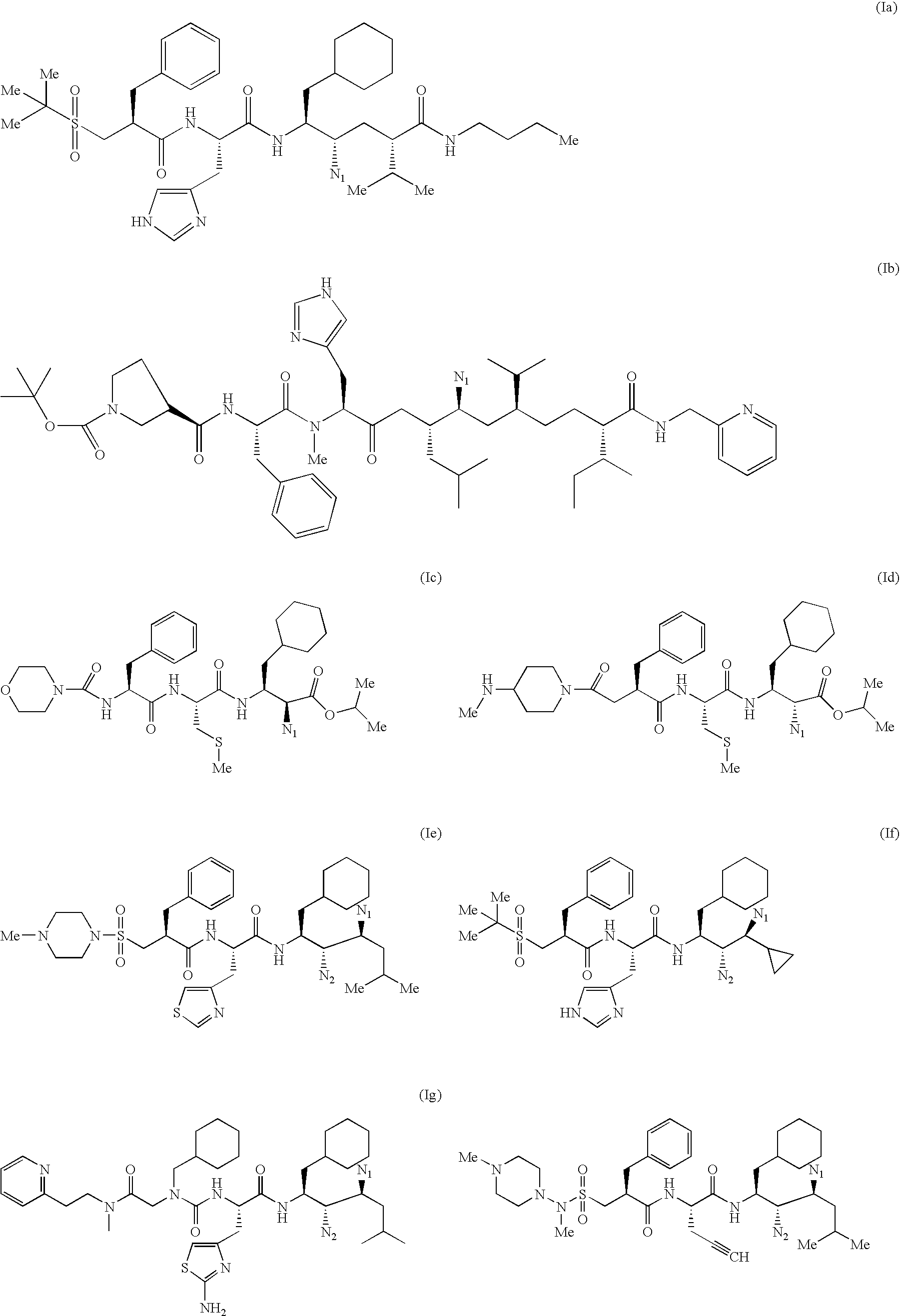

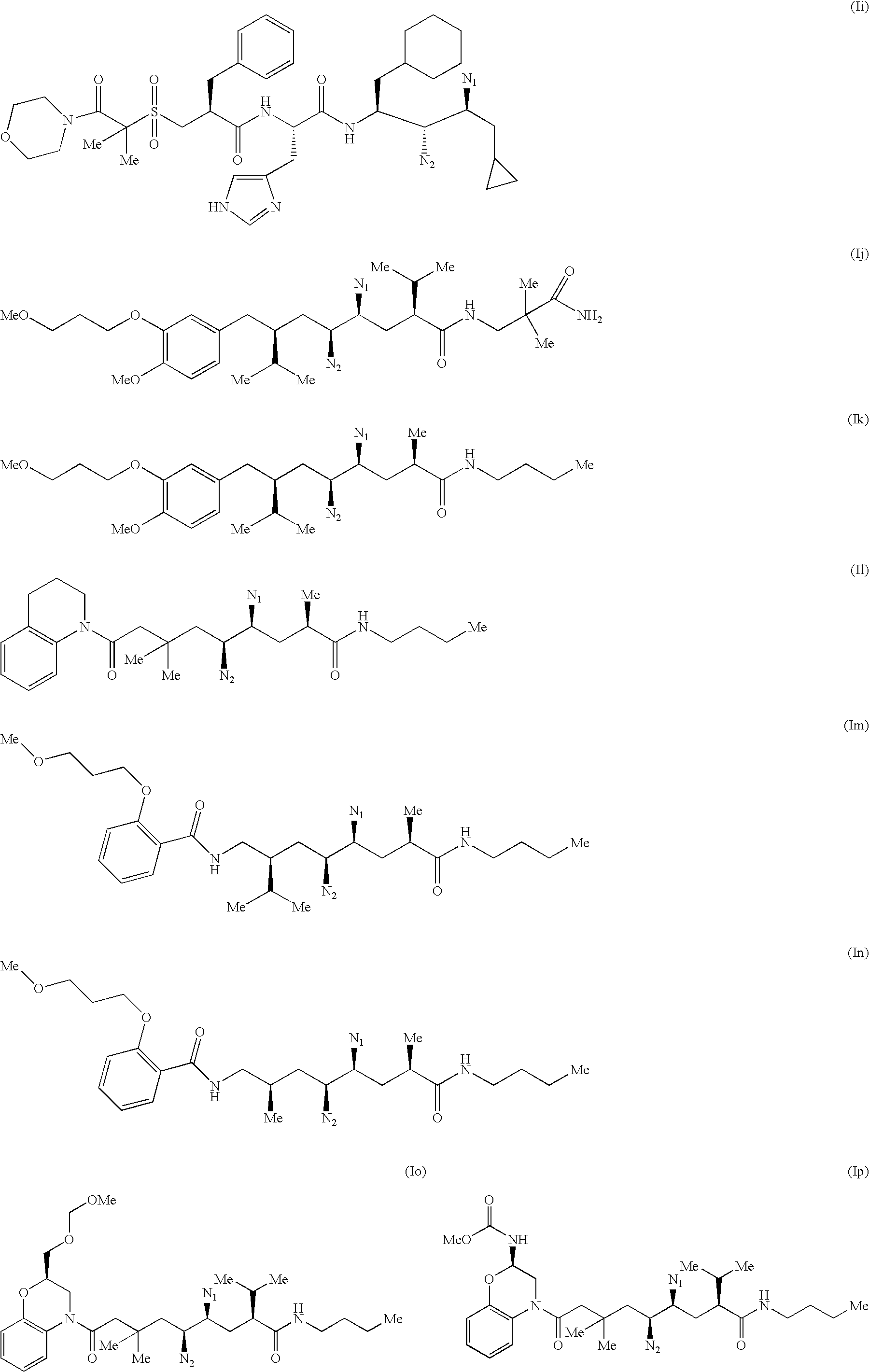

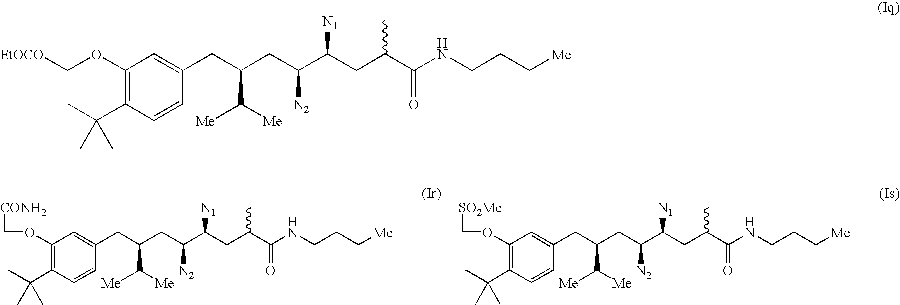

wherein:

### N₁ is —O— or —OH;

N₂ is —O—, —NH—, or N₃ wherein N₃ is —NH₂ or —OH;
With the proviso that at least one of N₁ and N₂ is a group —O— or —NH— able to bind to X₀;
X₀ is equal to —X₁—Y— wherein X₁ is —CO— or —COO—;
Y is a bivalent radical having the following meaning:
a)
straight or branched C₁-C₂₀ alkylene, preferably C₁-C₁₀, being optionally substituted with one or more of the substituents selected from the group consisting of: halogen atoms, hydroxy, —ONO₂ or T₀, wherein T₀ is —OC(O) (C₁-C₁₀ alkyl)-ONO₂ or —O(C₁-C₁₀ alkyl)-ONO₂;
cycloalkylene with 5 to 7 carbon atoms into cycloalkylene ring, the ring being optionally substituted with side chains T, wherein T is straight or branched alkyl with from 1 to 10 carbon atoms, preferably CH₃;
   b)

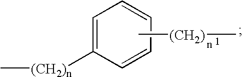

c)

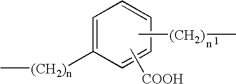

wherein n is an integer from 0 to 20, and n′ is an integer from 1 to 20;
d)

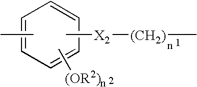

wherein:
n¹ is as defined above and n² is an integer from 0 to 2;

### X₂=—OCO— or —COO— and R² is H or CH₃;

e)

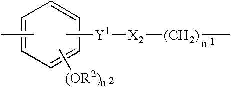

wherein:
n¹, n², R² and X₂ are as defined above;
Y¹ is —CH₂—CH₂— or —CH═CH— (CH₂)ₙ²—;
f)

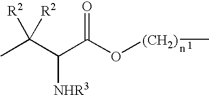

wherein:
n¹ and R² are as defined above, R³ is H or —COCH₃;
with the proviso that when Y is selected from the bivalent radicals mentioned under b)-f), the —ONO₂ group is linked to a —(CH₂)ₙ¹ group;
g)

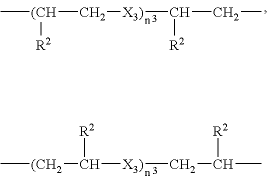

wherein X₃ is —O— or —S—, n³ is an integer from 1 to 6, preferably from 1 to 4, R² is as defined above;
h)

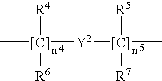

wherein:
n⁴ is an integer from 0 to 10;
n⁵ is an integer from 1 to 10;
R⁴, R⁵, R⁶, R⁷ are the same or different, and are H or straight or branched C₁-C₄ alkyl, preferably R⁴, R⁵, R⁶, R⁷ are H;
wherein the —ONO₂ group is linked to

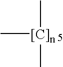

wherein n⁵ is as defined above;
Y² is an heterocyclic saturated, unsaturated or aromatic 5 or 6 members ring, containing one or more heteroatoms selected from nitrogen, oxygen, sulfur, and is selected from

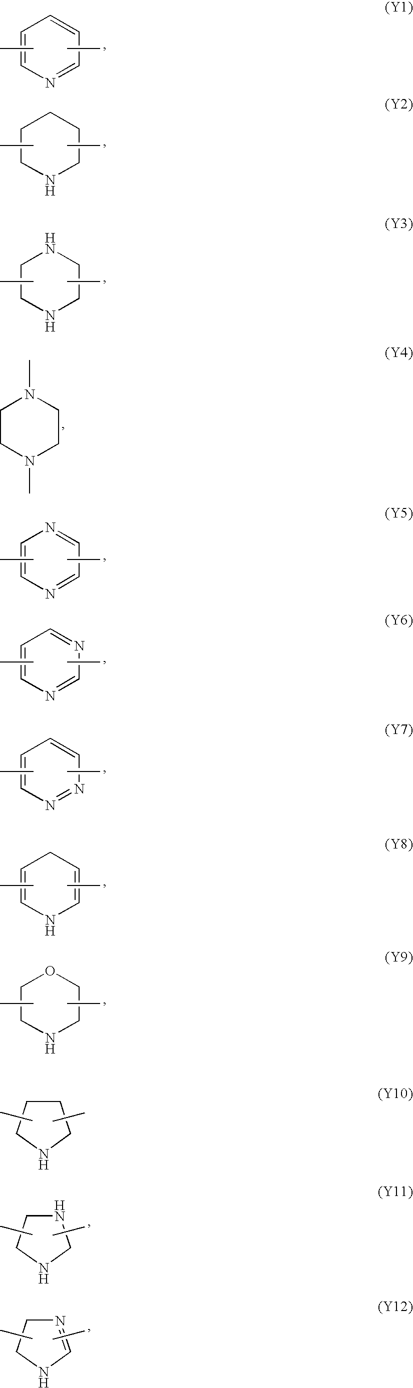

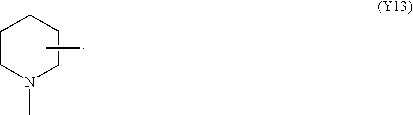

The term “C₁-C₂₀ alkylene” as used herein refers to branched or straight chain C₁-C₂₀ hydrocarbon, preferably having from 1 to 10 carbon atoms such as methylene, ethylene, propylene, isopropylene, n-butylene, pentylene, n-hexylene and the like.

The term “C₁-C₁₀ alkyl” as used herein refers to branched or straight chain alkyl groups comprising one to ten carbon atoms, including methyl, ethyl, n-propyl, isopropyl, n-butyl, isobutyl, t-butyl, pentyl, hexyl, octyl and the like.

The term “cycloalkylene” as used herein refers to ring having from 5 to 7 carbon atoms including, but not limited to, cyclopentylene, cyclohexylene optionally substituted with side chains such as straight or branched (C₁-C₁₀)-alkyl, preferably CH₃.

The term “heterocyclic” as used herein refers to saturated, unsaturated or aromatic 5 or 6 members ring, containing one or more heteroatoms selected from nitrogen, oxygen, sulphur, such as for example pyridine, pyrazine, pyrimidine, pyrrolidine, morpholine, imidazole and the like.

Another aspect of the present invention provides the use of the compounds of formula (I) in combination with at least a compound used to treat cardiovascular disease selected from the group consisting of: aldosterone antagonists, angiotensin II receptor blockers, ACE inhibitors, HMGCoA reductase inhibitors, beta-adrenergic blockers, alpha-adrenergic antagonists, sympatholitics, calcium channel blockers, endothelin antagonists, neutral endopeptidase inhibitors, potassium activators, diuretics, vasodilators, antithrombotics such as aspirin. Also is contemplated the combination with nitrosated compounds of the above reported compounds.

Suitable aldosterone antagonists, angiotensin II receptor blockers, ACE inhibitors, HMGCoA reductase inhibitors, beta-adrenergic blockers, alpha-adrenergic antagonists, calcium channel blockers, potassium activators, diuretics, vasodilators and antithrombotics are described in the literature such as The Merck Index (13^{th} edition).

Suitable nitrosated compounds are disclosed in WO 98/21193, WO 97/16405, WO 98/09948, WO 2004/105754, WO 2004/106300, WO 2004/110432, WO 2005/011646, WO 2005/053685, WO 2005/054218.

The administration of the compounds above reported can be carried out simultaneously or successively.

The present invention also provides pharmaceutical kits comprising one or more containers filled with one or more of the compounds and/or compositions of the present invention and one or more of the compounds used to treat cardiovascular diseases reported above.

As stated above, the invention includes also the pharmaceutically acceptable salts of the compounds of formula (I) and stereoisomers thereof.

Examples of pharmaceutically acceptable salts are either those with inorganic bases, such as sodium, potassium, calcium and aluminium hydroxides, or with organic bases, such as lysine, arginine, triethylamine, dibenzylamine, piperidine and other acceptable organic amines.

The compounds according to the present invention, when they contain in the molecule one salifiable nitrogen atom, can be transformed into the corresponding salts by reaction in an organic solvent such as acetonitrile, tetrahydrofuran with the corresponding organic or inorganic acids.

Examples of organic acids are: oxalic, tartaric, maleic, succinic, citric acids. Examples of inorganic acids are: nitric, hydrochloric, sulphuric, phosphoric acids. Salts with nitric acid are preferred.

The compounds of the invention which have one or more asymmetric carbon atoms can exist as optically pure enantiomers, pure diastereomers, enantiomers mixtures, diastereomers mixtures, enantiomer racemic mixtures, racemates or racemate mixtures. Within the object of the invention are also all the possible isomers, stereoisomers and their mixtures of the compounds of formula (I).

Preferred compounds are those of formula (I) wherein Y has the following meaning:

a)
straight or branched C₁-C₁₀ alkylene;
   b)

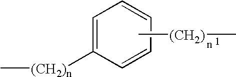

wherein n is 0 or 1, n¹ is 1;
with the proviso that the —ONO₂ group is linked to —(CH₂)ₙ¹ group;
g)

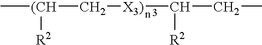

wherein X₃ is —O— or —S—, n³ is 1 and R² is H;

The following are preferred compounds according to the present invention:

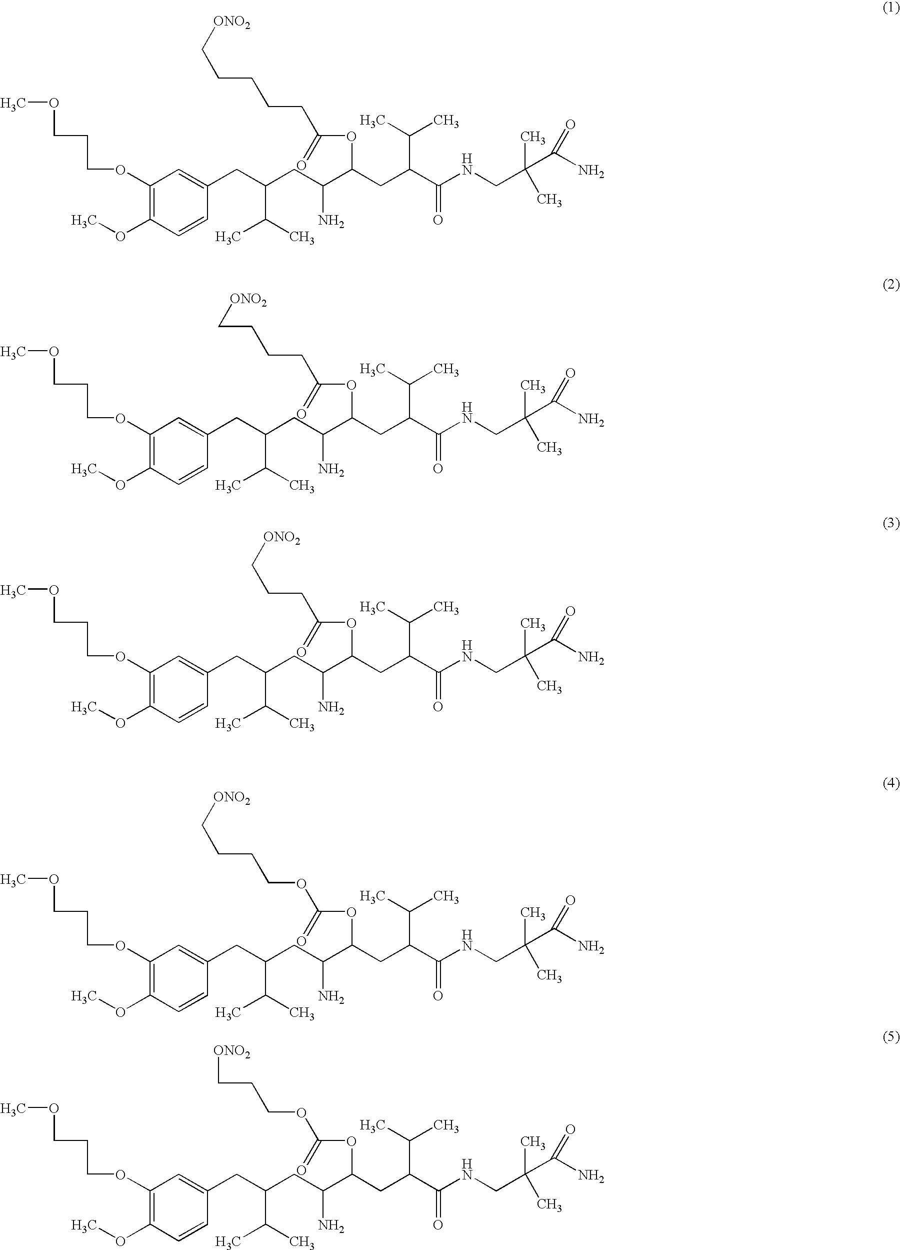

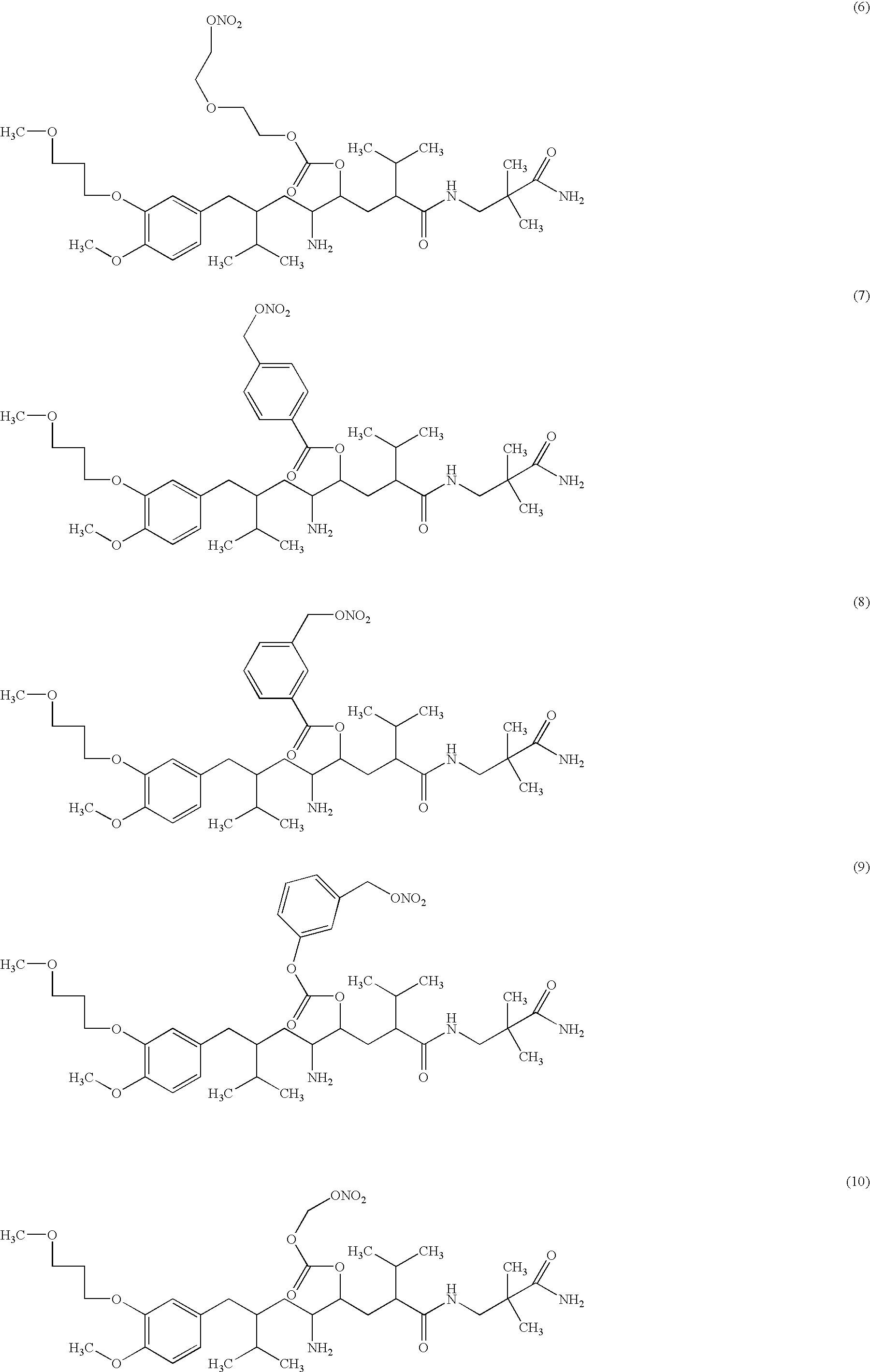

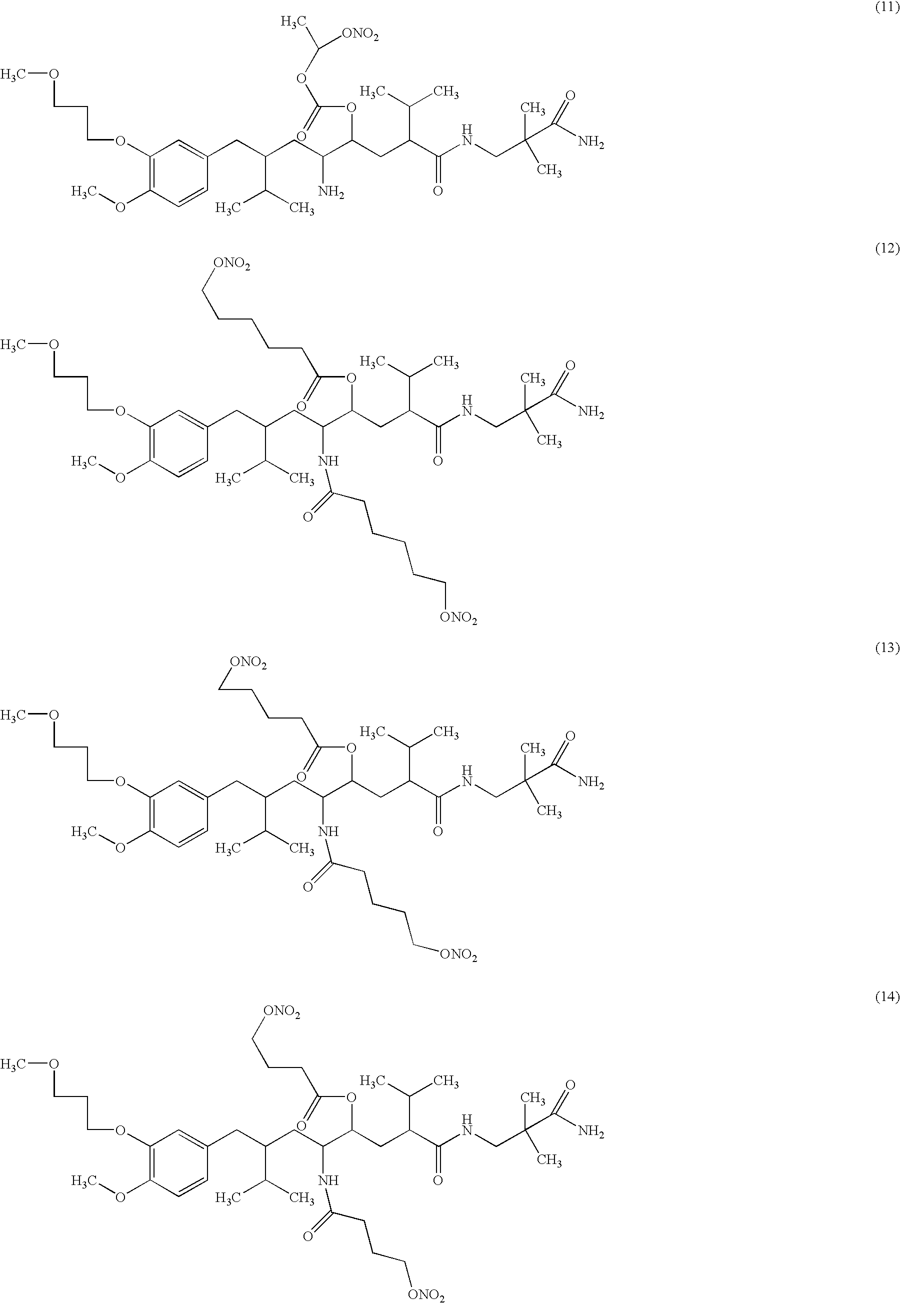

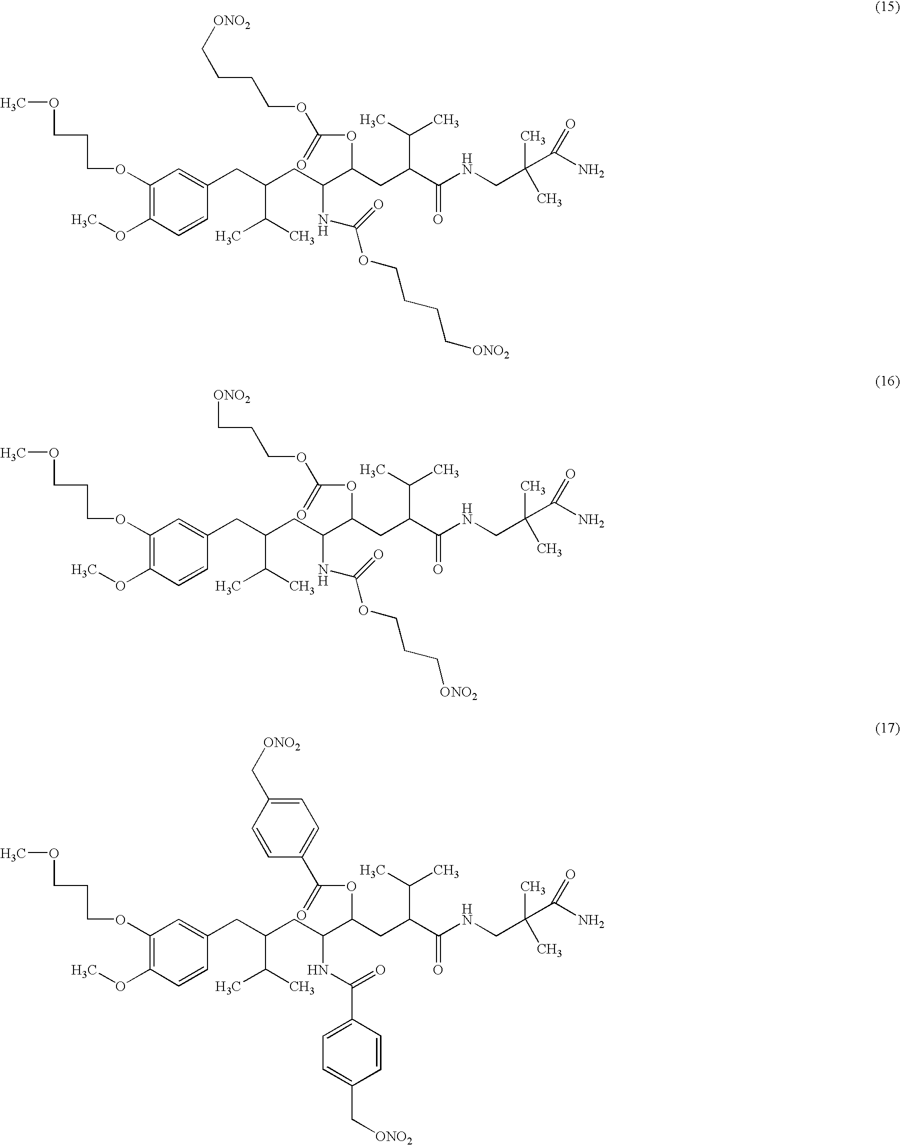

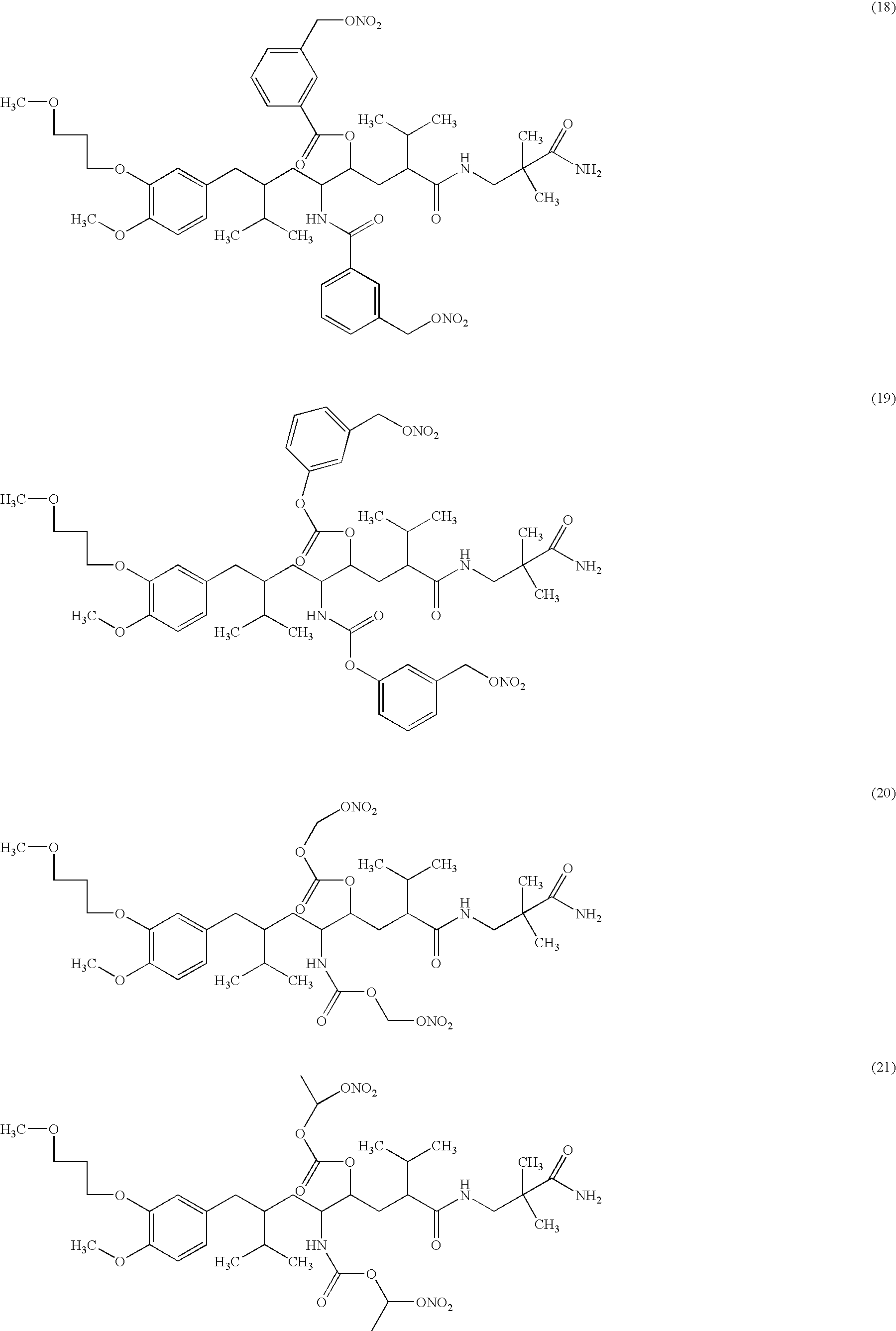

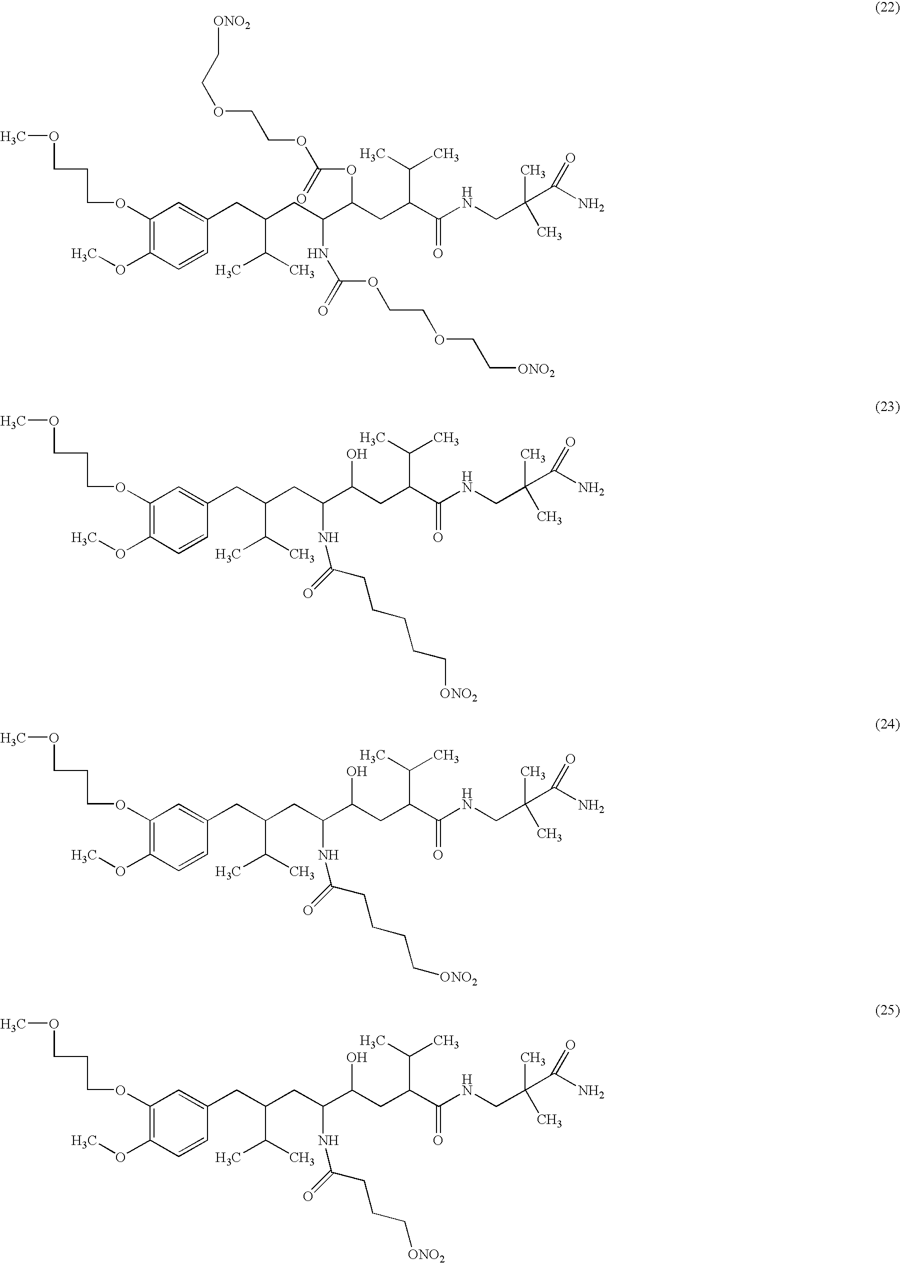

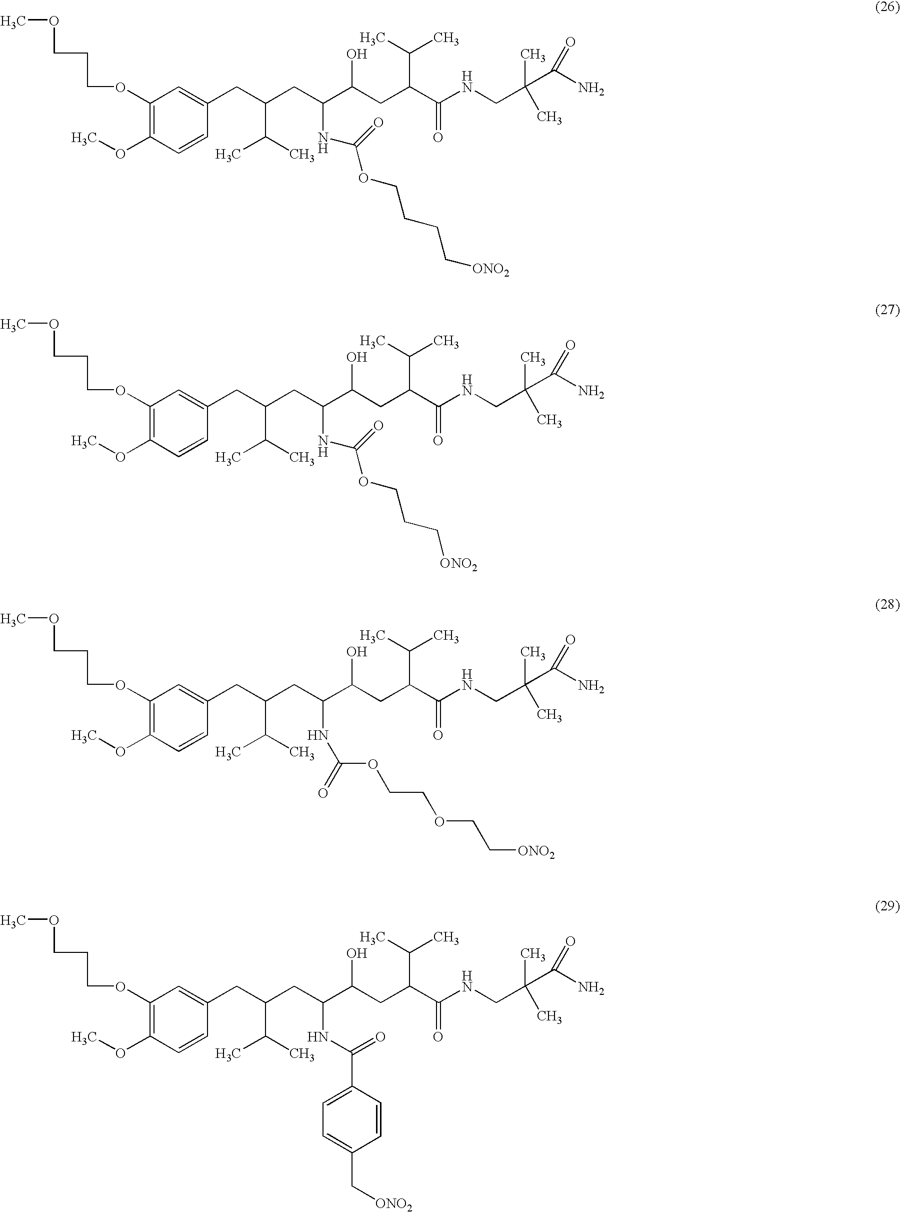

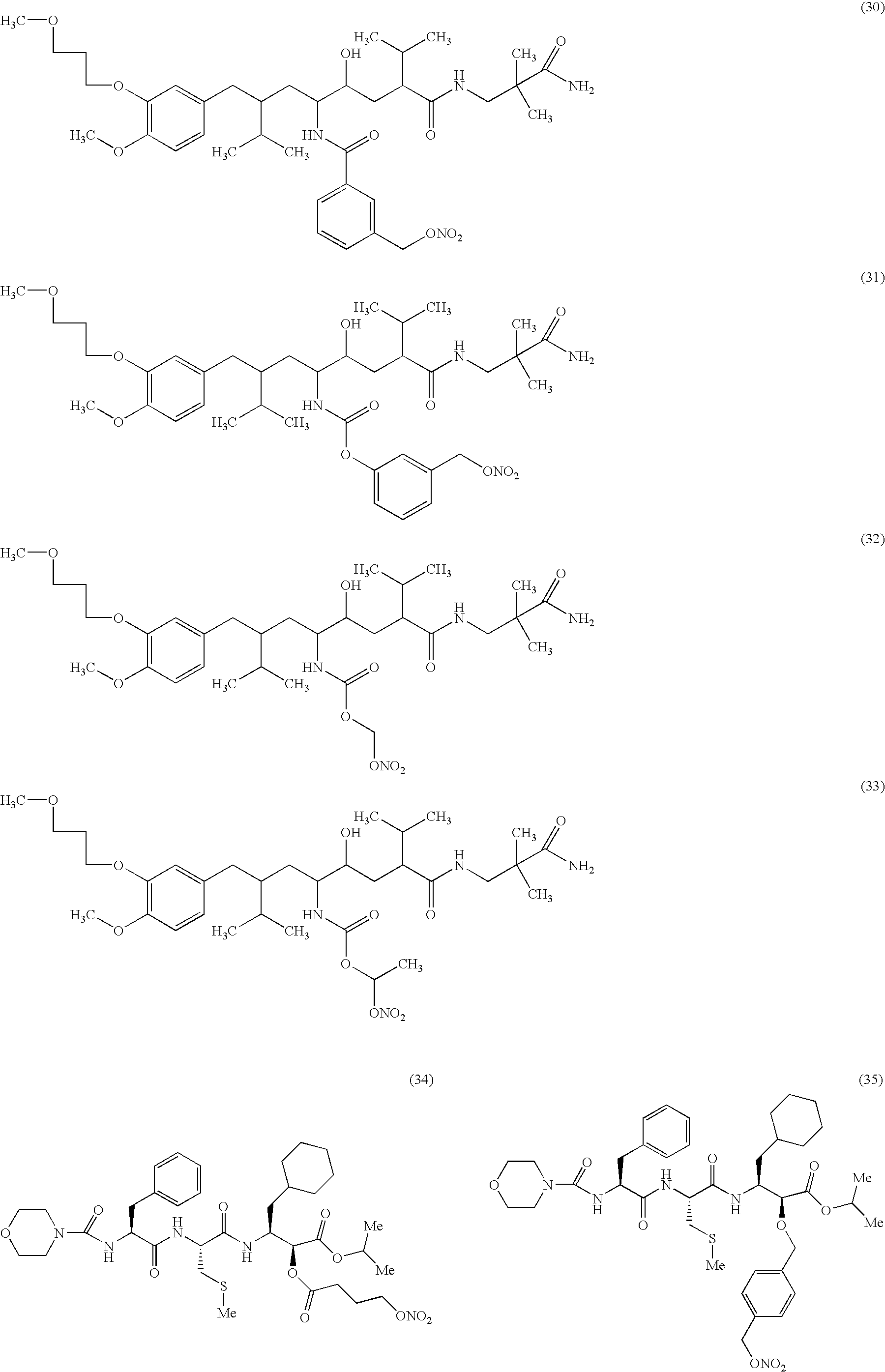

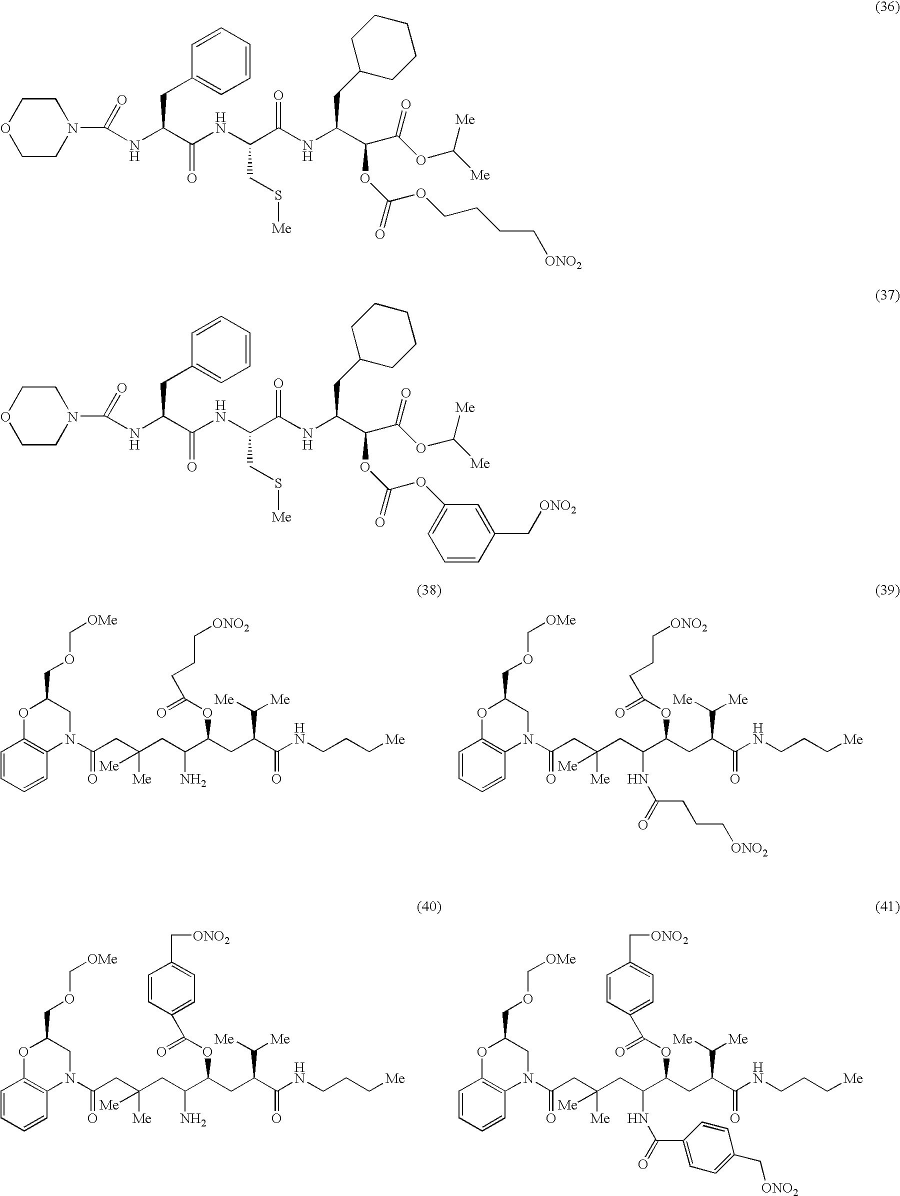

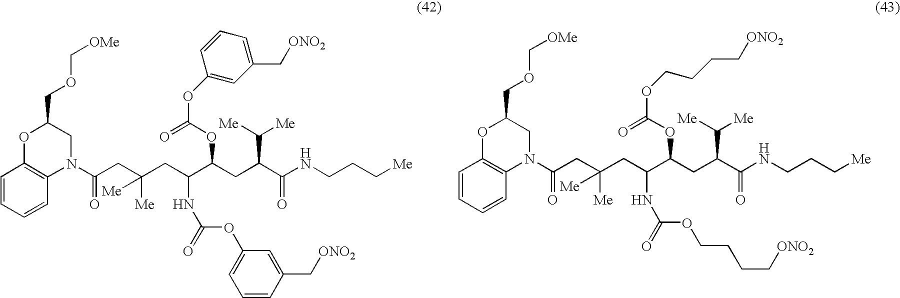

As mentioned above, object of the present invention are also pharmaceutical compositions containing at least a compound of the present invention of formula (I) together with non toxic adiuvants and/or carriers usually employed in the pharmaceutical field.

The daily dose of active ingredient that should be administered can be a single dose or it can be an effective amount divided into several smaller doses that are to be administered throughout the day. Usually, total daily dose may be in amounts preferably from 50 to 500 mg. The dosage regimen and administration frequency for treating the mentioned diseases with the compound of the invention and/or with the pharmaceutical compositions of the present invention will be selected in accordance with a variety of factors, including for example age, body weight, sex and medical condition of the patient as well as severity of the disease, route of administration, pharmacological considerations and eventual concomitant therapy with other drugs. In some instances, dosage levels below or above the aforesaid range and/or more frequent may be adequate, and this logically will be within the judgment of the physician and will depend on the disease state.

The compounds of the invention may be administered orally, parenterally, rectally or topically, by inhalation or aerosol, in formulations eventually containing conventional non-toxic pharmaceutically acceptable carriers, adjuvants and vehicles as desired. Topical administration may also involve the use of transdermal administration such as transdermal patches or iontophoresis devices. The term “parenteral” as used herein, includes subcutaneous injections, intravenous, intramuscular, intrasternal injection or infusion techniques.

Injectable preparations, for example sterile injectable aqueous or oleaginous suspensions may be formulated according to known art using suitable dispersing or wetting agents and suspending agents. The sterile injectable preparation may also be a sterile injectable solution or suspension in a non-toxic parenterally acceptable diluent or solvent. Among the acceptable vehicles and solvents are water, Ringer's solution and isotonic sodium chloride. In addition, sterile, fixed oils are conventionally employed as a solvent or suspending medium. For this purpose any bland fixed oil may be employed including synthetic mono or diglycerides, in addition fatty acids such as oleic acid find use in the preparation of injectables.

Suppositories for rectal administration of the drug can be prepared by mixing the active ingredient with a suitable non-irritating excipient, such as cocoa butter and polyethylene glycols.

Solid dosage forms for oral administration may include capsules, tablets, pills, powders, granules and gels. In such solid dosage forms, the active compound may be admixed with at least one inert diluent such as sucrose, lactose or starch. Such dosage forms may also comprise, as in normal practice, additional substances other than inert diluents, e.g. lubricating agents such as magnesium stearate. In the case of capsules, tablets and pills, the dosage forms may also comprise buffering agents. Tablets and pills can additionally be prepared with enteric coatings.

Liquid dosage forms for oral administration may include pharmaceutically acceptable emulsions, solutions, suspensions, syrups and elixirs containing inert diluents commonly used in the art, such as water. Such compositions may also comprise adjuvants, such as wetting agents, emulsifying and suspending agents, and sweetening, flavouring and the like.

The compounds of the present invention can be synthesized as follows.

### Synthesis Procedure

1. The compound of general formula (I) as above defined wherein:
s is 1; X₀ is —X₁—Y— wherein X₁ is —CO— and Y is as above defined and A is selected among compounds (Ia-Ic), can be obtained by a process comprising:
1a. reacting a compound of formula B with a compound of formula (IIIa):

B+HOOC—Y—ONO₂ (IIIa)

wherein Y is as above defined; B is equal to A with A selected among (Ia-Ic) and N₁ is —OH; in presence of a condensing agent like dicyclohexylcarbodiimide (DCC) or N,N′-carbonyldiimidazol (CDI) or other known condensing reagents such as HATU in solvent such as DMF, THF, chloroform at a temperature in the range from −5° C. to 50° C. in the presence or not of a base as for example DMAP.

The nitric acid ester compounds of formula (IIIa) can be obtained from the corresponding alcohols of formula HOOC—Y—OH (IIIb), that are commercially available, by reaction with nitric acid and acetic anhydride in a temperature range from −50° C. to 0° C. or reacting the corresponding halogen derivatives of formula HOOC—Y-Hal (IIIc) wherein Hal is an alogen atom preferable Cl, Br, I, that are commercially available, with AgNO₃ as already described in the international application No. PCT/EP2005/050459.

Compound of formula B wherein B is equal to A when A is (Ia) wherein N₁ is —OH is a known compound named CGP 38560 and can be prepared as described in Buehlmayer, P. et al. J. Med. Chem. 1988, 31, 1839.

Compound of formula B wherein B is equal to A when A is (Ib) wherein N, is —OH is a known compound named ditekiren and can be prepared as described in U.S. Pat. No. 4,880,781.

Compound of formula B wherein B is equal to A when A is (Ic) wherein N₁ is —OH is a known compound named terlakiren and can be prepared as described in ES2061512T.

1a.1 reacting a compound of formula B as above defined with a compound of formula (IIId):

B+Act-CO—Y—ONO₂ (IIId)

wherein Y is as above defined; Act is an Halogen atom or a carboxylic acid activating group used in peptide chemistry as:

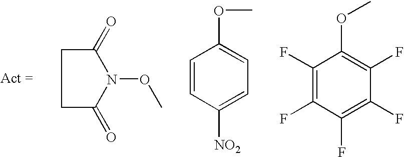

The reaction is generally carried out in presence of a inorganic or organic base in an aprotic polar/non-polar solvent such as DMF, THF or CH₂Cl₂ at temperatures range between 0°-65° C. or in a double phase system H₂O/Et₂O at temperatures range between 20°-40° C.; or in the presence of DMAP and a Lewis acid such as Sc(OTf)₃ or Bi(OTf)₃ in solvents such as DMF, CH₂Cl₂.

The compounds of formula (IIId) can be obtained as described in the international application No. PCT/EP2005/050459.

1a.2 reacting a compound of formula A-X₀-Hal (IVa), wherein A and X₀ are as above defined, with AgNO₃ as already described. Compounds (IVa) can be obtained by reacting compound B with compounds (IIIc), as above defined, with a condensing reagent such as DCC or CDI as above described.
1a.3 reacting a compound of formula A-X₀—OH (Va), wherein A and X₀ are as above defined, with triflic anhydride/tetraalkylammonium nitrate salt in an aprotic polar/non-polar solvent such as DMF, THF or CH₂Cl₂ at temperatures range between −60° to 65° C. as already described. Compounds (Va) can be obtained by reacting compound B with compounds (IIIb), as above defined, with a condensing reagent as above described.
2. The compound of general formula (I) as above defined wherein:
s is 1; X₀ is —X₁—Y— wherein X₁ is —C(O)O— and Y is as above defined and A is selected among compounds (Ia-Ic), can be obtained by a process comprising:
2a. reacting a compound of formula B with a compound of formula (VIa):

B+Act-CO—O—Y—ONO₂ (VIa)

wherein B, Act and Y are as above described.

The reaction is generally carried out in presence of a inorganic or organic base in an aprotic polar/non-polar solvent such as DMF, THF or CH₂Cl₂ at temperatures range between 0°-65° C. or in a double phase system H₂O/Et₂O at temperatures range between 20°-40° C.; or in the presence of DMAP and a Lewis acid such as Sc(OTf)₃ or Bi(OTf)₃ in solvents such as DMF, CH₂Cl₂.

The synthesis of compounds (VIa) has already been described in the international application No. PCT/EP2005/050459.

2a.1 reacting a compound of formula A-X₀-Hal (VIIa) wherein X₀, A and Hal are as above defined, with AgNO₃ as above described.

The compounds of formula (VIIa) can be obtained by reacting compound B with compounds Act-CO—O—Y-Hal (VIIb). The reaction is generally carried out in presence of an inorganic or organic base in an aprotic polar/non-polar solvent such as DMF, THF or CH₂Cl₂ at temperatures range between 0°-65° C. as above described.

Compound (VIIb) are commercially available or can be synthesized as already described in WO 05/011646.

3. The compound of general formula (I) as above defined or a pharmaceutically acceptable salt thereof, wherein:
s is 1; X₀ is —X₁—Y— wherein X₁ is —CO— or —C(O)O and Y is as above defined and A is compound (Id), can be obtained by a process comprising:
3a. when X₁ is —CO—, reacting a compound of formula C

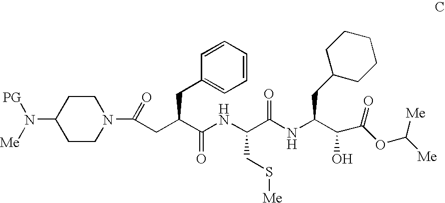

wherein PG is an amine protective group such as BOC with compounds of formula (IIIa) or (IIId) as above defined, or when X₁ is —C(O)O— with compounds of formula (VIa) with the same procedure already described for compounds of formula B, and eventually acid hydrolysing the N—BOC protective group as known in the literature and salifying if required.

Compound C can be obtained from compound D, known in the literature as CP 108,671 prepared as described in EP0661292.

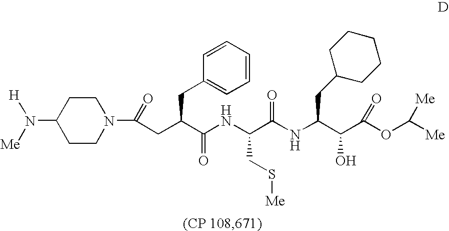

by reacting compound D with (BOC)₂O and TEA or with other well known methods as described in T. W. Greene “Protective groups in organic synthesis”, Harvard University Press, 1980.
4. The compound of general formula (I) as above defined wherein:
s is 2; X₀ is —X₁—Y— wherein X₁ is —CO— or —C(O)O— and Y is as above defined and A is selected among compounds (Ie-Ii), can be obtained by a process comprising:
4a. reacting a compound of formula E wherein E is equal to A with A selected among (Ie-Ii) with N₁ is —OH and N₂ is equal to N₃ where N₃ is —OH with excess of a compound of formula (IIIa), or (IIId) or (VIa) as above described for analogous reactions of compounds B and C.

Compound of formula E wherein E is equal to A when A is (Ie) wherein N₁ is —OH and N₂ is equal to N₃ where N₃ is —OH is a known compound named zankiren and can be prepared as described in Rosenberg, S. H. et al. J. Med. Chem. 1993, 36, 460.

Compound of formula E wherein E is equal to A when A is (If) wherein N₁ is —OH and N₂ is equal to N₃ where N₃ is —OH is a known compound named remikiren and can be prepared as described in EP0509354.

Compound of formula E wherein E is equal to A when A is (Ig) wherein N₁ is —OH and N₂ is equal to N₃ where N₃ is —OH is a known compound named BILA 2157 BS and can be prepared as described in Beaulieu P. L. et al. J. Org. Chem. 1999, 64, 6622.

Compound of formula E wherein E is equal to A when A is (Ih) wherein N₁ is —OH and N₂ is equal to N₃ where N₃ is —OH is a known compound named SC 56525 and can be prepared as described in EP0655063.

Compound of formula E wherein E is equal to A when A is (Ii) wherein N₁ is —OH and N₂ is equal to N₃ where N₃ is —OH is a known compound named ciprokiren and can be prepared as described in EP0509354.

5. The compound of general formula (I) as above defined wherein:
s is 1; X₀ is —X₁—Y— wherein X₁ is —CO— and Y is as above defined and A is selected among compounds (Ij-Is), wherein N₁ is —OH and N₂ is equal to —NH— can be obtained by a process comprising:
5a. reacting a compound of formula F with the stoichiometric amount of a compound of formula (IIIa) as above defined;

F is equal to A with A selected among (Ij-Is) with N₁ equal to —OH and N₂ equal to N₃ where N₃ is —NH₂, in presence of a condensing agent like dicyclohexylcarbodiimide (DCC) or N,N′-carbonyldiimidazol (CDI) or other known condensing reagents such as HATU in solvent such as DMF, THF, chloroform at a temperature in the range from −5° C. to 50° C. in the presence or not of a base as for example TEA, DMAP. Compound of formula F wherein F is equal to A when A is (Ij) wherein N₁ is —OH and N₂ is equal to N₃ where N₃ is —NH₂ is a known compound named aliskiren or SPP100 and can be prepared as described in U.S. Pat. No. 5,559,111.

Compound of formula F wherein F is equal to A when A is (Ik) wherein N₁ is —OH and N₂ is equal to N₃ where N₃ is —NH₂ is a known compound and can be prepared as described in U.S. Pat. No. 5,559,111.

Compound of formula F wherein F is equal to A when A is (Il) wherein N₁ is —OH and N₂ is equal to N₃ where N₃ is —NH₂ is a known compound and can be prepared as described in EP 702004.

Compound of formula F wherein F is equal to A when A is (Im) wherein N₁ is —OH and N₂ is equal to N₃ where N₃ is —NH₂ is a known compound and can be prepared as described in EP 716077.

Compound of formula F wherein F is equal to A when A is (In) wherein N₁ is —OH and N₂ is equal to N₃ where N₃ is —NH₂ is a known compound and can be prepared as described in EP 716077.

Compound of formula F wherein F is equal to A when A is (Io) wherein N₁ is —OH and N₂ is equal to N₃ where N₃ is —NH₂ is a known compound named CGP 56346A and can be prepared as described in Maibaum J. et al., “Design and synthesis of novel potent, non-peptide and orally active renin inhibitors”, Medicinal Chemistry: Today and Tomorrow, Proceedings of the AFMC International Medicinal Chemistry Symposium, Tokyo, Sep. 3-8, 1995.

Compound of formula F wherein F is equal to A when A is (Ip) wherein N₁ is —OH and N₂ is equal to N₃ where N₃ is —NH₂ is a known compound named CGP 55128A and can be prepared as described in Maibaum J. et al., “Design and synthesis of novel potent, non-peptide and orally active renin inhibitors”, Medicinal Chemistry: Today and Tomorrow, Proceedings of the AFMC International Medicinal Chemistry Symposium, Tokyo, Sep. 3-8, 1995.

Compound of formula F wherein F is equal to A when A is (Iq) wherein N₁ is —OH and N₂ is equal to N₃ where N₃ is —NH₂ is a known compound and can be prepared as described in Göschke, R: et al. Bioorg. Med. Chem. Lett. 1997, 7, 2735.

Compound of formula F wherein F is equal to A when A is (Ir) wherein N₁ is —OH and N₂ is equal to N₃ where N₃ is —NH₂ is a known compound and can be prepared as described in Göschke, R: et al. Bioorg. Med. Chem. Lett. 1997, 7, 2735. Compound of formula F wherein F is equal to A when A is (Is) wherein N₁ is —OH and N₂ is equal to N₃ where N₃ is —NH₂ is a known compound and can be prepared as described in Göschke, R: et al. Bioorg. Med. Chem. Lett. 1997, 7, 2735.

5a.1 reacting a compound of formula F where F is as above defined with the stoichiometric amount of a compound of formula (IIId) as above defined, using the procedure already described for compound B.
5a.2 reacting a compound of formula A-X₀-Hal (IVa), wherein X₀ is as above defined and A is selected among (Ij-Is) with N₁ equal to —OH and N₂ is —NH, with AgNO₃ as previously described.

Compounds (IVa) wherein X₀ is as above defined and A is selected among (Ij-s) with N₁ equal to N₃ where N₃ is —OH and N₂ is —NH can be synthesized from compounds of formula F where F is as above defined with stoichiometric amount of compound (IIIc) and a condensing agent as already described.

6. The compound of general formula (I) as above defined wherein:
s is 1; X₀ is —X₁—Y— wherein X₁ is —C(O)O— and Y is as above defined and A is selected among compounds (Ij-Is), wherein N₁ is —OH and N₂ is —NH— can be obtained by a process comprising:
6a. reacting a compound of formula F with the stoichiometric amount of a compound of formula (VIa) as above defined, using the same procedure above described for compound B.
6a.1 reacting a compound of formula A-X₀-Hal (VIIa) wherein X₀ is —X₁—Y— wherein X₁ is —C(O)O— and Y is as above defined and A is selected among compounds (Ij-Is), wherein N₁ is —OH and N₂ is —NH, Hal is as above defined, with AgNO₃. Compounds (VIIa) can be synthesized from compounds of formula F, where F is as above defined, with compound (VIIb) as already described.
7. The compound of general formula (I) as above defined or a pharmaceutically acceptable salt thereof, wherein:
s is 1; X₀ is —X₁—Y— wherein X₁ is —CO— or —C(O)O and Y is as above defined and A is selected among (Ij-Is), where N₁ is —O— and N₂ is equal to N₃ where N₃ is —NH₂ can be obtained by a process comprising:
7a. removing the protective group from a compound of formula G-X₀—ONO₂ (VIIIa), wherein X₀ is —X₁—Y— wherein X₁ is —CO— or —C(O)O and Y is as above defined and G is equal to A when A is selected among (Ij-Is) where N₁ is —O— and N₂ is equal to N₄ where N₄ is —NH-PG where PG is an amino protective group like BOC or other, as above defined, with methods known in the literature and eventually salifying with a pharmaceutically acceptable acid.

Compounds of formula (VIIIa), when X₁ is —CO—, can be obtained from compounds of formula H wherein H is equal to A when A is selected among (Ij-Is) where N₁ is —OH and N₂ is equal to N₄ where N₄ is —NH-PG where PG is an amino protective group like BOC by reacting compounds H with compounds of formula (IIIa) or (IIId); or when X₁ is —C(O)O— with compounds of formula (VIa) using the same procedures already described for B.

7a.1 reacting a compound of formula G-X₀—OH (IXa) wherein G is as above defined, X₀ is —X₁—Y— wherein X₁ is —CO— and Y is as above defined, with triflic anhydride/tetraalkylammonium nitrate salt, eventually removing the protective group with methods known in the literature and if desired salifying with a pharmaceutically acceptable acid. Compounds (IXa) can be obtained by reacting compound of formula H defined above with compounds (IIIb) with a condensing reagent as above described.
8. The compound of general formula (I) as above defined wherein:
s is 2; X₀ is —X₁—Y— wherein X₁ is —CO— or —C(O)O— and Y is as above defined and A is selected among compounds (Ij-Is), wherein N₁ is —O— and N₂ is —NH— can be obtained by a process comprising:
8a. reacting a compound of formula F, when X₁ is —CO—, with excess amount of a compound of formula (IIIa) or (IIId); or when X₁ is —C(O)O— with excess amount of a compound of formula (VIa), using the same procedures above described for compound B.

8a.1 reacting a compound of formula A-(X₀-Hal)₂ (IXa) wherein X₀ is —X₁—Y— wherein X₂ is —CO— or —C(O)O— and Y is as above defined and A is selected among compounds (Ij-Is), wherein N₁ is —O— and N₂ is —NH—, Hal is as above defined, with AgNO₃.

Compounds (IXa) wherein A and X₀ are as above defined can be synthesized from compounds of formula F where F is as above defined with excess of compound (IIIc) or (VIIb) as already described.

8a.2 reacting a compound of formula G-(X₀—OH)₂ (Xa) wherein G is as above defined, wherein X₀ is —X₁—Y— wherein X₁ is —CO— and Y is as above defined with triflic anhydride/tetraalkylammonium nitrate salt as already described. Compounds (Xa) can be obtained by reacting compound of formula H defined above with excess of compounds (IIIb) with a condensing reagent as above described.

The following examples are to further illustrate the invention without limiting it.

### EXAMPLE 1

### Synthesis of (2S,4S,5S,7S)-5-[4-(nitrooxy)butanoyl]amino-4-hydroxy-2-isopropyl-7-[4-methoxy-3-(3-methoxy-propopxy)-benzyl]-8-methyl-nonanoic acid (2-carbamoyl-2-methyl-propyl)-amide (corresponding to formula 25)

To a solution of (2S,4S,5S,7S)-5-amino-4-hydroxy-2-isopropyl-7-[4-methoxy-3-(3-methoxy-propoxy)-benzyl]-8-methyl-nonanoic acid (2-carbamoyl-2-methyl-propyl)-amide (aliskiren) (0.347 g, 0.630 mmol) TEA (0.064 g, 0.630 mmol) and DMAP (0.077 g, 0.630 mmol) in DMF (10 ml) kept at 0° C., under stirring and under nitrogen atmosphere, a solution of 4-(nitrooxy)butanoic acid pentafluorophenyl ester (0.20 g, 0.630 mmol) in DMF (2 ml) is added. The resulting solution is kept under stirring for further 240 minutes at room temperature. The reaction mixture is poured in a pH 3 buffer solution (about 50 ml), acidified with HCl 1 N to pH 2-3 and extracted with CH₂Cl₂ (2×50 ml). The organic phase is washed with brine (100 ml), dried on sodium sulfate and evaporated under vacuum.

The crude product is purified by flash chromatography (CH₂Cl₂: MeOH 9:1 as eluant) to give the title compound (0.215 g, 50%).

### EXAMPLE 2

### Synthesis of (2S,4S,5S,7S)-5-[4-(nitrooxy)butanoyl]amino-4-[4-(nitrooxy)butanoyl]oxy-2-isopropyl-7-[4-methoxy-3-(3-methoxy-propopxy)-benzyl]-8-methyl-nonanoic acid (2-carbamoyl-2-methyl-propyl)-amide (corresponding to formula 14)

To a solution of (2S,4S,5S,7S)-5-amino-4-hydroxy-2-isopropyl-7-[4-methoxy-3-(3-methoxy-propoxy)-benzyl]-8-methyl-nonanoic acid (2-carbamoyl-2-methyl-propyl)-amide (aliskiren) (0.347 g, 0.630 mmol) TEA (0.128 g, 1.26 mmol) and DMAP (0.154 g, 1.26 mmol) in DMF (10 ml) kept at 0° C., under stirring and under nitrogen atmosphere, a solution of 4-(nitrooxy)butanoic acid pentafluorophenyl ester (0.40 g, 1.26 mmol) in DMF (4 ml) is added. The resulting solution is kept under stirring for further 240 minutes at room temperature. The reaction mixture is poured in a pH 3 buffer solution (about 50 ml), acidified with HCl 1 N to pH 2-3 and extracted with CH₂Cl₂ (2×50 ml). The organic phase is washed with brine (100 ml), dried on sodium sulfate and evaporated under vacuum.

The crude product is purified by flash chromatography (CH₂Cl₂: MeOH 9:1 as eluant) to give the title compound (0.204 g, 45%).

### EXAMPLE 3

### Synthesis of (2S,4S,5S,7S)-5-[[[4-(nitrooxy)butyl]oxy]carbonyl]amino-4-hydroxy-2-isopropyl-7-[4-methoxy-3-(3-methoxy-propopxy)-benzyl]-8-methyl-nonanoic acid (2-carbamoyl-2-methyl-propyl)-amide (corresponding to formula 26)

To a solution of (2S,4S,5S,7S)-5-amino-4-hydroxy-2-isopropyl-7-[4-methoxy-3-(3-methoxy-propoxy)-benzyl]-8-methyl-nonanoic acid (2-carbamoyl-2-methyl-propyl)-amide (aliskiren) (0.347 g, 0.630 mmol) TEA (0.064 g, 0.630 mmol) and DMAP (0.077 g, 0.630 mmol) in DMF (10 ml) kept at 0° C., under stirring and under nitrogen atmosphere, a solution of 4-(nitrooxy)butyl p-nitrophenyl carbonate (0.190 g, 0.630 mmol) in DMF (2 ml) is added. The resulting solution is kept under stirring for further 240 minutes at room temperature. The reaction mixture is poured in a pH 3 buffer solution (about 50 ml), acidified with HCl 1 N to pH 2-3 and extracted with CH₂Cl₂ (2×50 ml). The organic phase is washed with brine (100 ml), dried on sodium sulfate and evaporated under vacuum.

The crude product is purified by flash chromatography (CH₂Cl₂: MeOH 9:1 as eluant) to give the title compound (0.209 g, 50%).

### EXAMPLE 4

### Synthesis of (2S,4S,5S,7S)-5-[4-[(nitrooxy)methyl]benzoyl]amino-4-hydroxy-2-isopropyl-7-[4-methoxy-3-(3-methoxy-propopxy)-benzyl]-8-methyl-nonanoic acid (2-carbamoyl-2-methyl-propyl)-amide (corresponding to formula 29)

To a solution of (2S,4S,5S,7S)-5-amino-4-hydroxy-2-isopropyl-7-[4-methoxy-3-(3-methoxy-propoxy)-benzyl]-8-methyl-nonanoic acid (2-carbamoyl-2-methyl-propyl)-amide (aliskiren) (0.347 g, 0.630 mmol), Sc(OTf)₃ (0.03 g, 0.061 mmol) and DMAP (0.154 g, 1.26 mmol) in DMF (10 ml) kept at 0° C., under stirring and under nitrogen atmosphere, a solution of [4-(nitrooxy)methyl]benzoic acid pentafluorophenyl ester (0.20 g, 0.630 mmol) in DMF (2 ml) is added. The resulting solution is kept under stirring for further 240 minutes at room temperature. The reaction mixture is poured in a pH 3 buffer solution (about 50 ml), acidified with HCl 1 N to pH 2-3 and extracted with CH₂Cl₂ (2×50 ml). The organic phase is washed with brine (100 ml), dried on sodium sulfate and evaporated under vacuum. The crude product is purified by flash chromatography (CH₂Cl₂: MeOH 9:1 as eluant) to give the title compound (0.130 g, 30%).

1. A compound of general formula (I) or a pharmaceutically acceptable salt or stereoisomer thereof:
A-(X₀—ONO₂)ₛ (I)
wherein:
s is an integer equal to 1 or 2;
A is selected from the following groups: 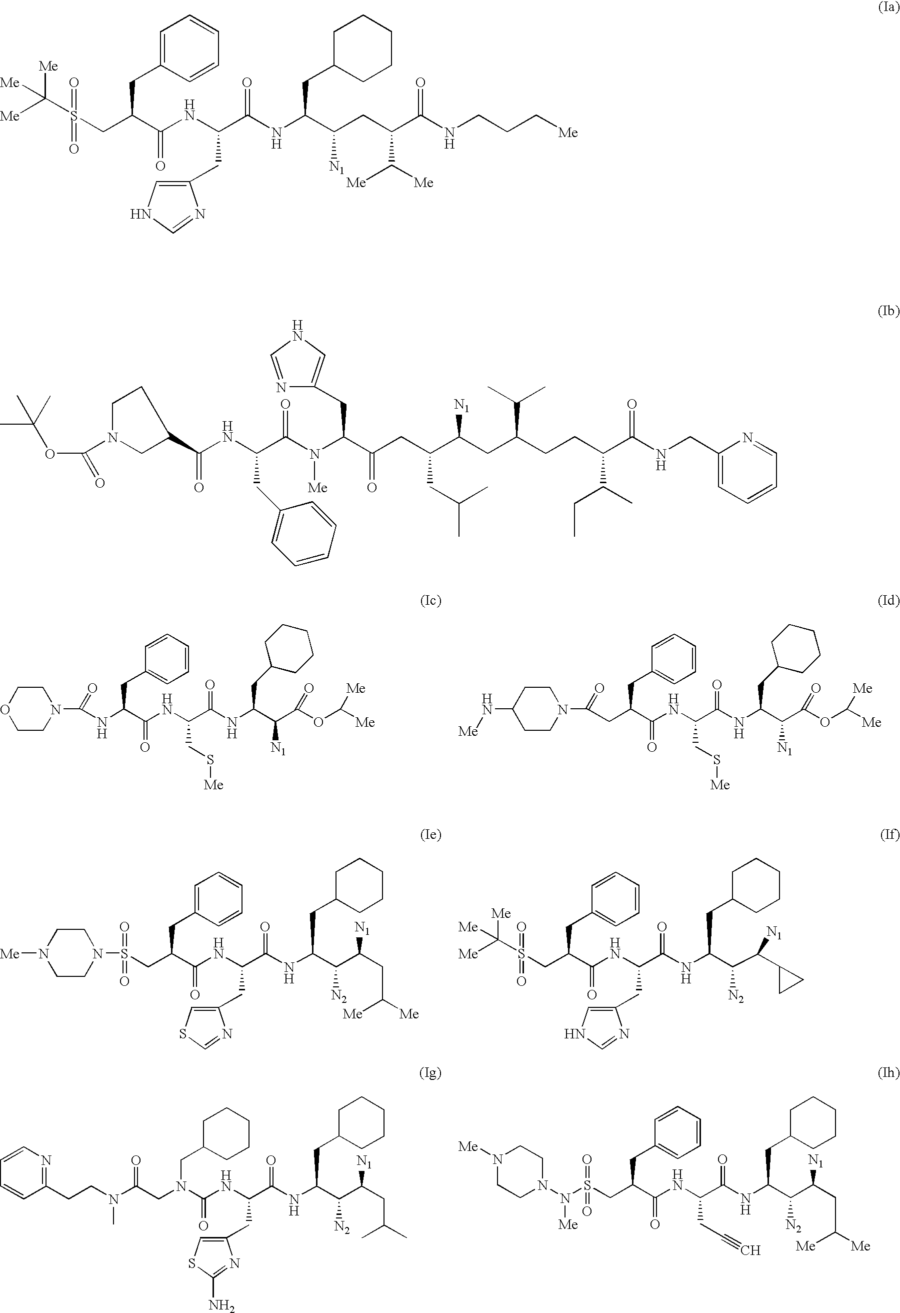 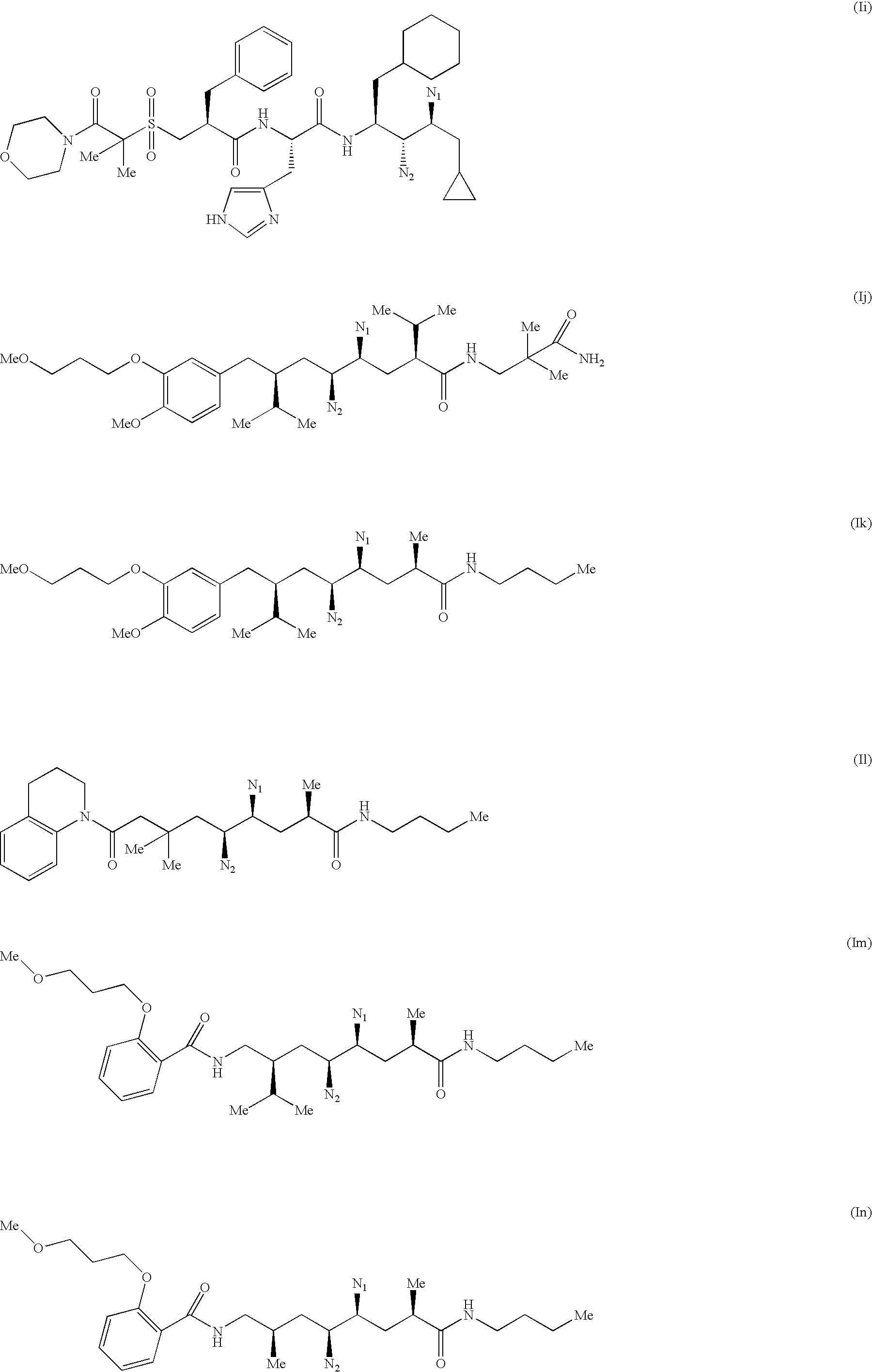 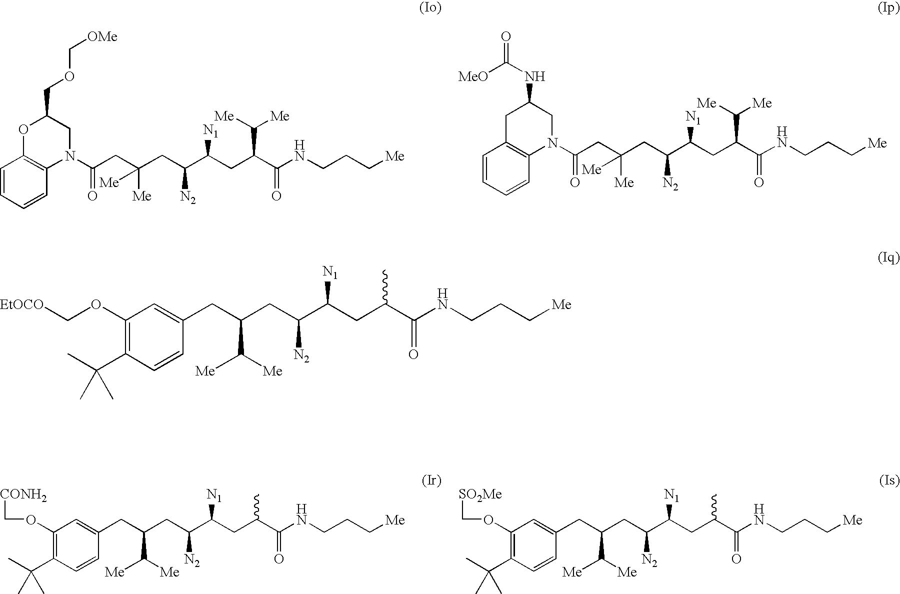
wherein:
N₁ is —O— or —OH;
N₂ is —O—, —NH—, or N₃ wherein N₃ is —NH₂ or —OH;
With the proviso that at least one of N₁ and N₂ is a group —O— or —NH— able to bind to X₀;
X₀ is equal to —X₁—Y— wherein X₁ is —CO— or —COO—;
Y is a bivalent radical having the following meaning:
a)
straight or branched C₁-C₂₀ alkylene, preferably C₁-C₁₀, being optionally substituted with one or more of the substituents selected from the group consisting of: halogen atoms, hydroxy, —ONO₂ or T₀, wherein T₀ is
OC(O)(C₁-C₁₀ alkyl)-ONO₂ or —O(C₁-C₁₀ alkyl)-ONO₂;
cycloalkylene with 5 to 7 carbon atoms into cycloalkylene ring, the ring being optionally substituted with side chains T, wherein T is straight or branched alkyl with from 1 to 10 carbon atoms, preferably CH₃;
b) 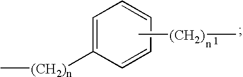
c) 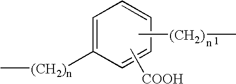
wherein n is an integer from 0 to 20, and n¹ is an integer from 1 to 20;
d) 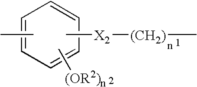
wherein:
n¹ is as defined above and n² is an integer from 0 to 2;
X₂=—OCO— or —COO— and R² is H or CH₃;
e) 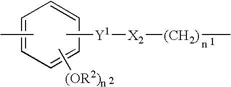
wherein:
n¹, n², R² and X₂ are as defined above;
Y¹ is —CH₂—CH₂— or —CH═CH—(CH₂)ₙ²—;
f) 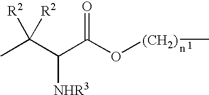
wherein:
n¹ and R² are as defined above, R³ is H or —COCH₃;
with the proviso that when Y is selected from the bivalent radicals mentioned under b)-f), the —ONO₂ group is linked to a —(CH₂)ₙ¹ group;
g) 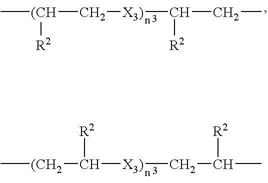
wherein X₃ is —O— or —S—, n³ is an integer from 1 to 6, preferably from 1 to 4, R² is as defined above; 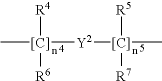
wherein:
n⁴ is an integer from 0 to 10;
n⁵ is an integer from 1 to 10;
R⁴, R⁵, R⁶, R⁷ are the same or different, and are H or straight or branched C₁-C₄ alkyl, preferably R⁴, R⁵, R⁶, R⁷ are H;
wherein the —ONO₂ group is linked to 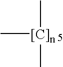
wherein n5 is as defined above;
Y² is an heterocyclic saturated, unsaturated or aromatic 5 or 6 members ring, containing one or more heteroatoms selected from nitrogen, oxygen, sulfur, and is selected from

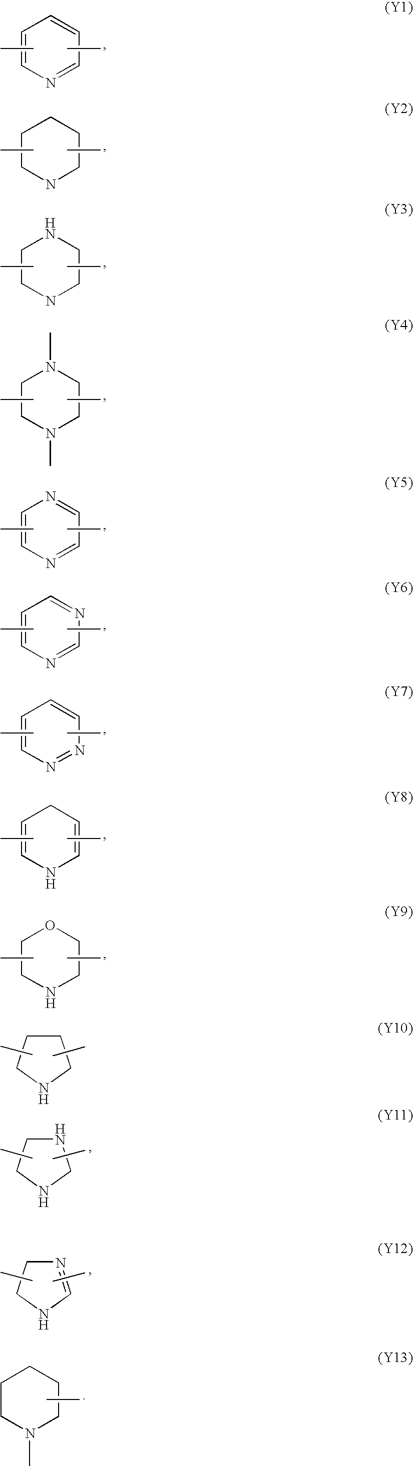

2. A compound of general formula (I) or a pharmaceutically acceptable salt or stereoisomer thereof according to claim 1 wherein Y is a bivalent radical having the following meaning:
a)
straight or branched C₁-C₁₀ alkylene;
b) 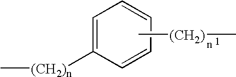
wherein n is 0 or 1, n¹ is 1;
with the proviso that the —ONO₂ group is linked to —(CH₂)ₙ₁ group;
g) 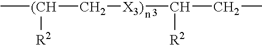
wherein X₃ is —O— or —S—, n³ is 1 and R² is H;

3. A compound according to claim 1, selected from the group consisting of:

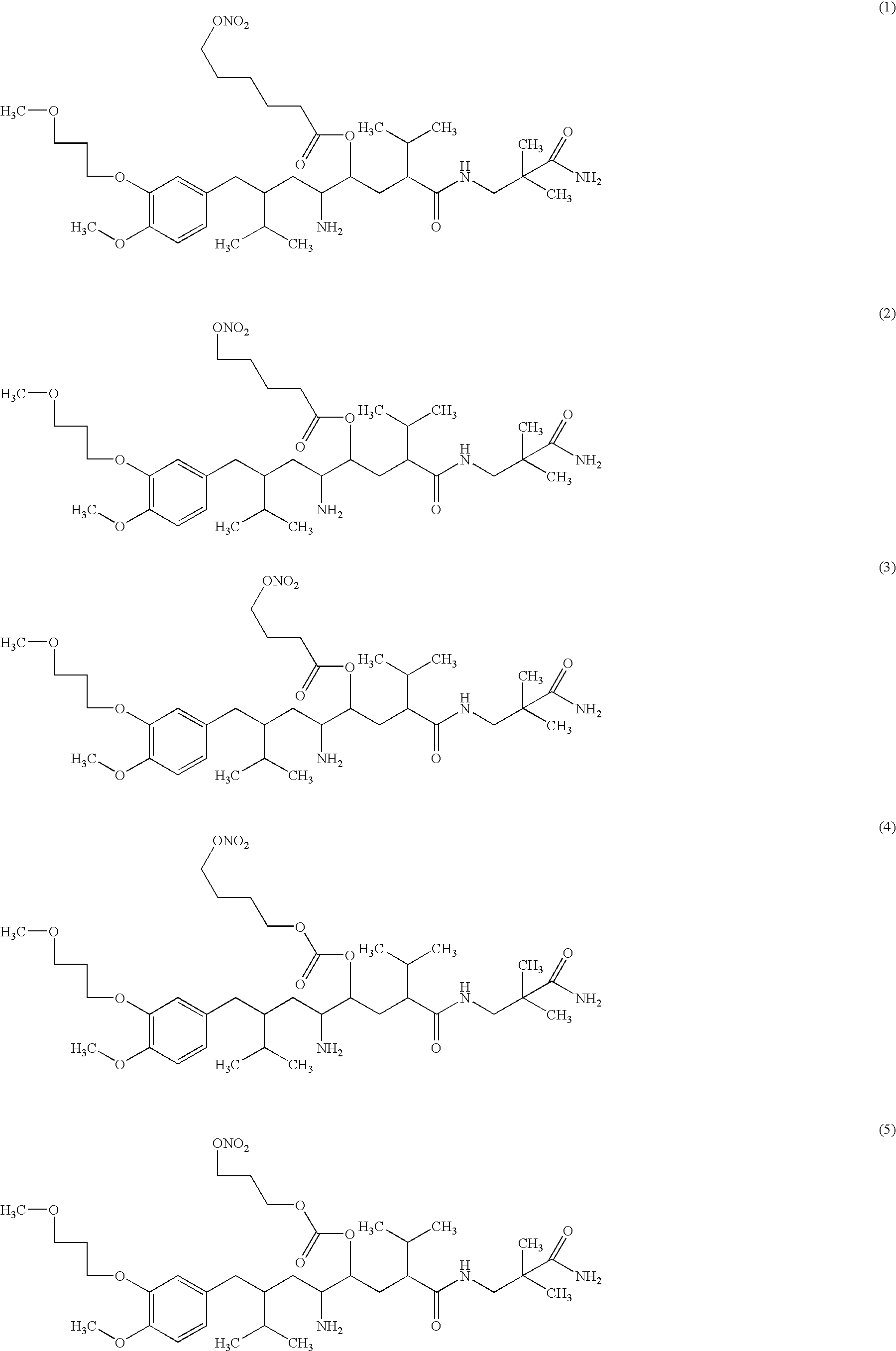

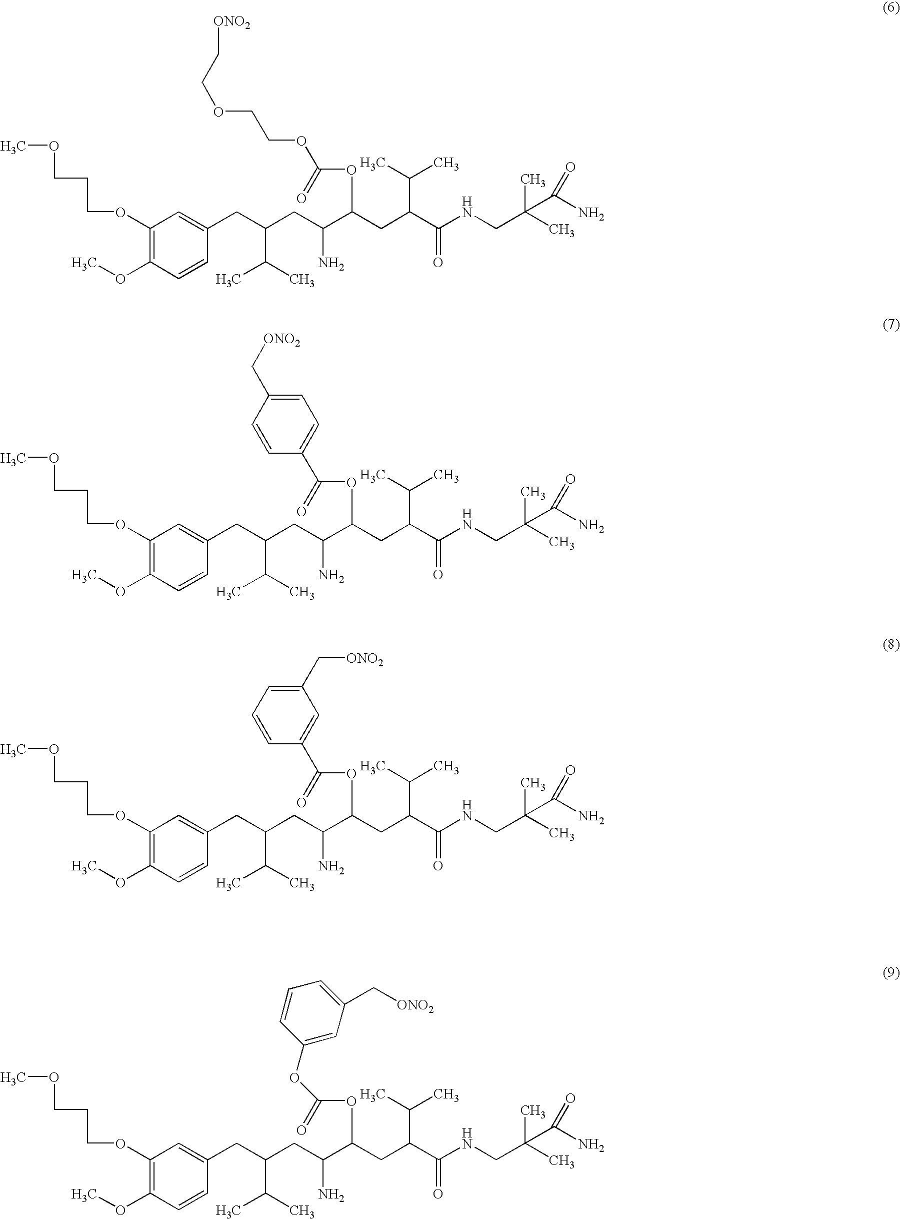

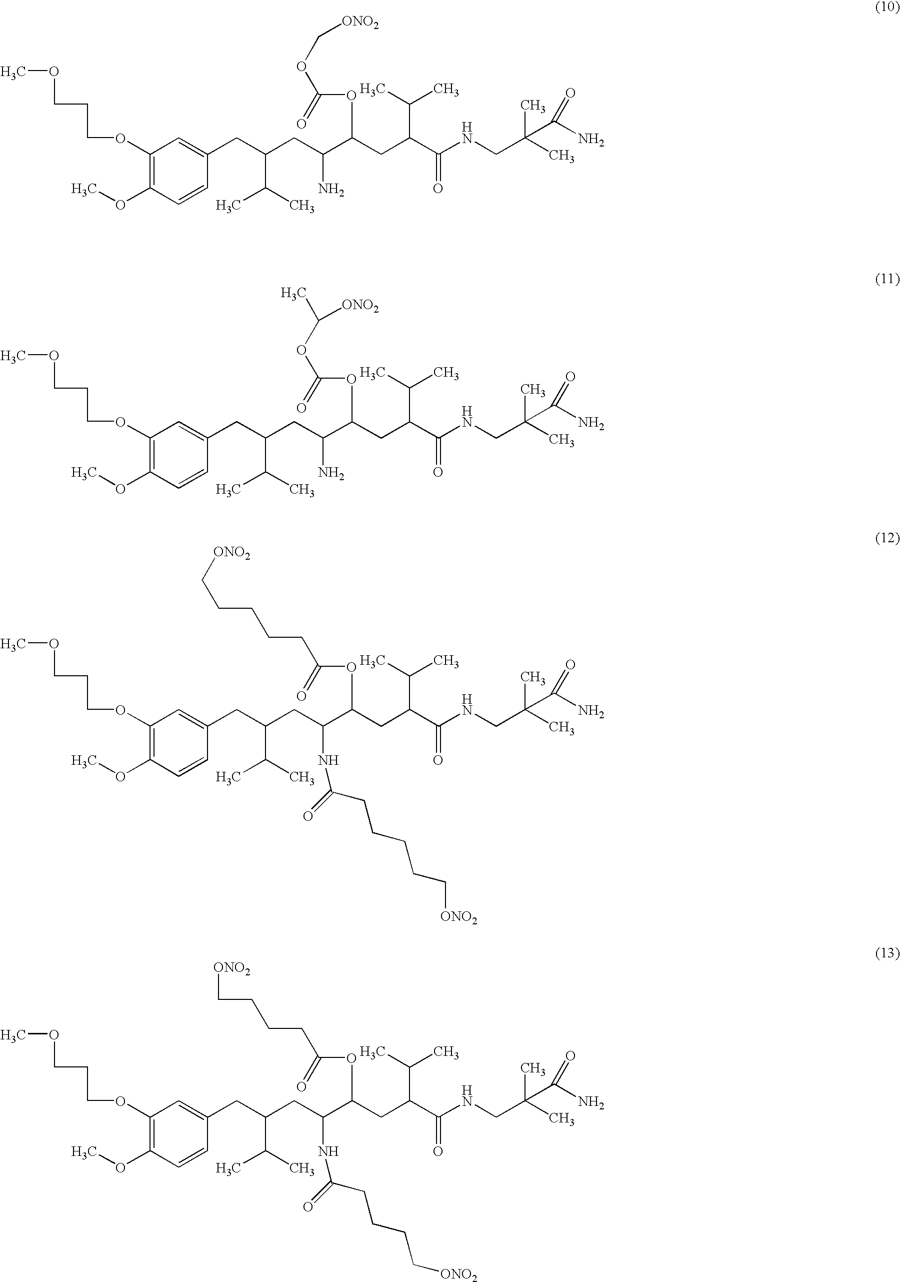

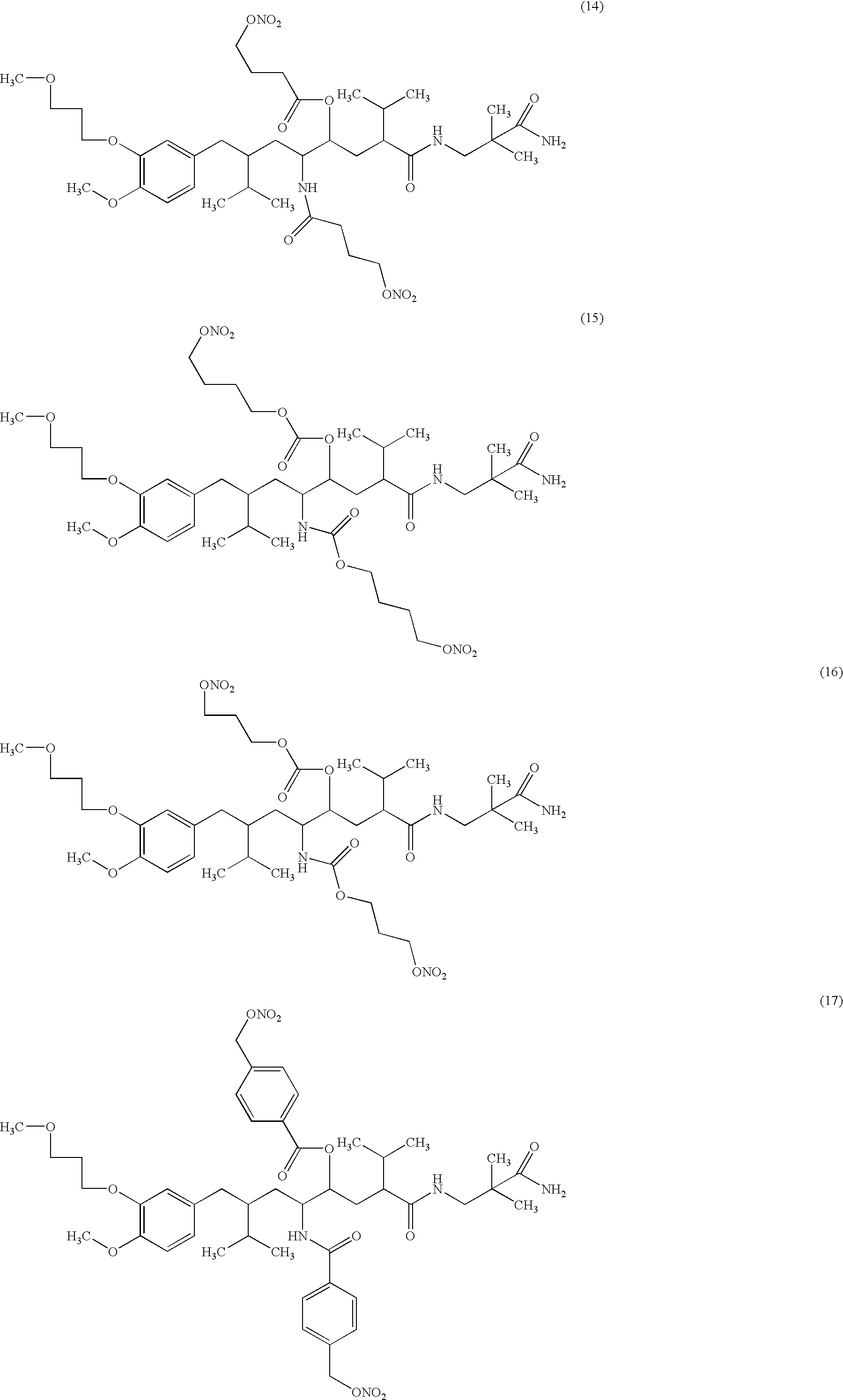

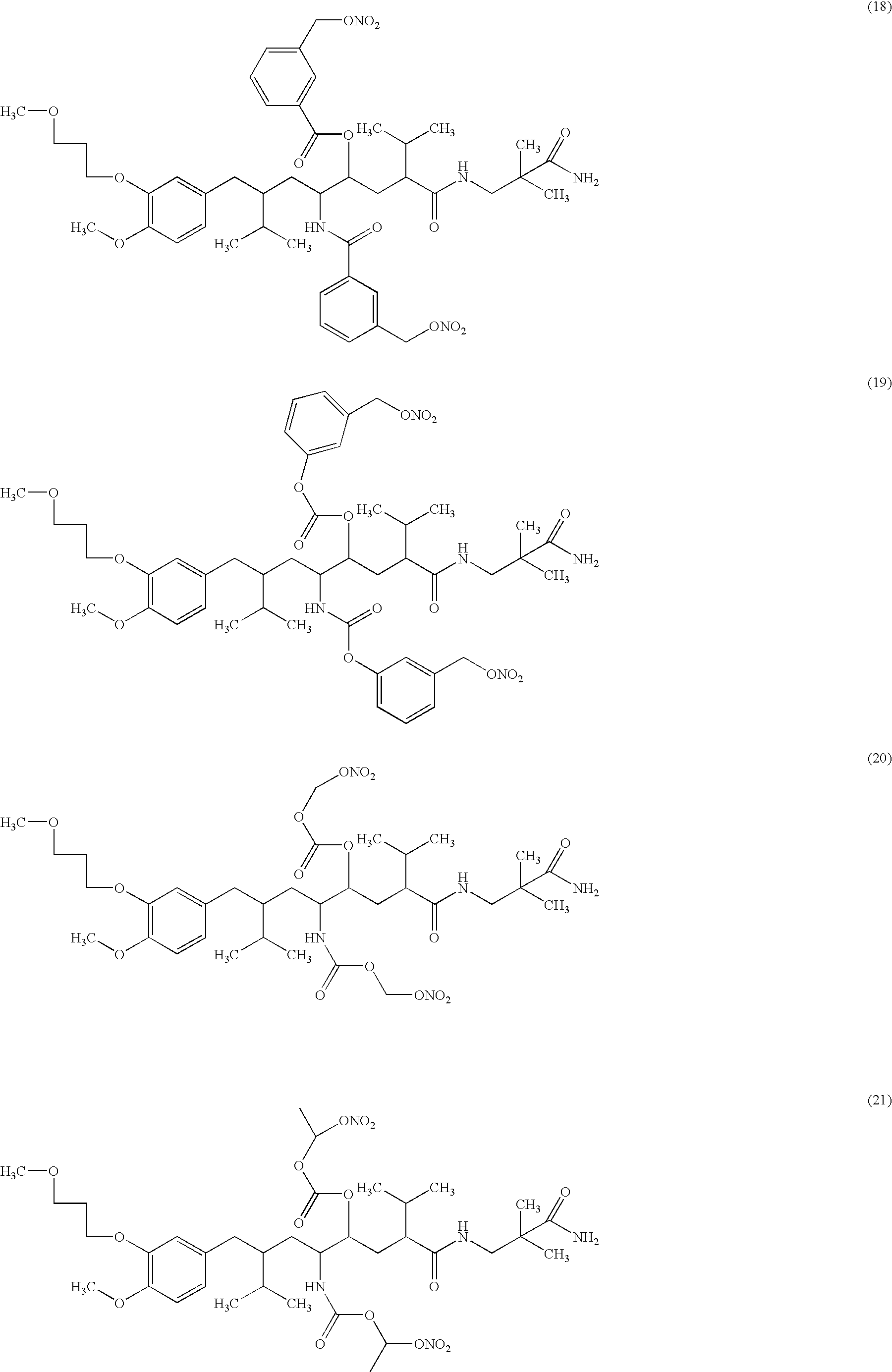

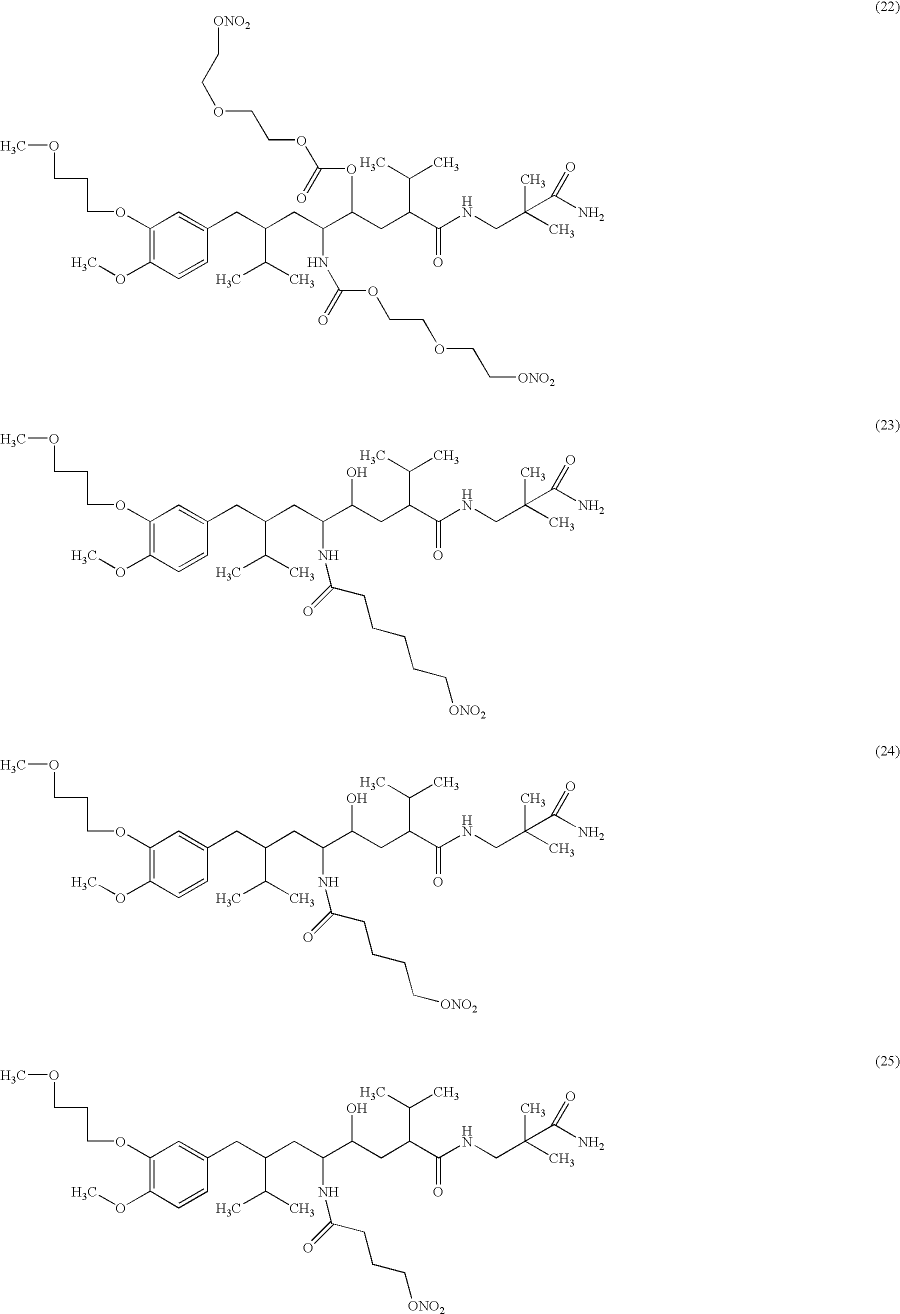

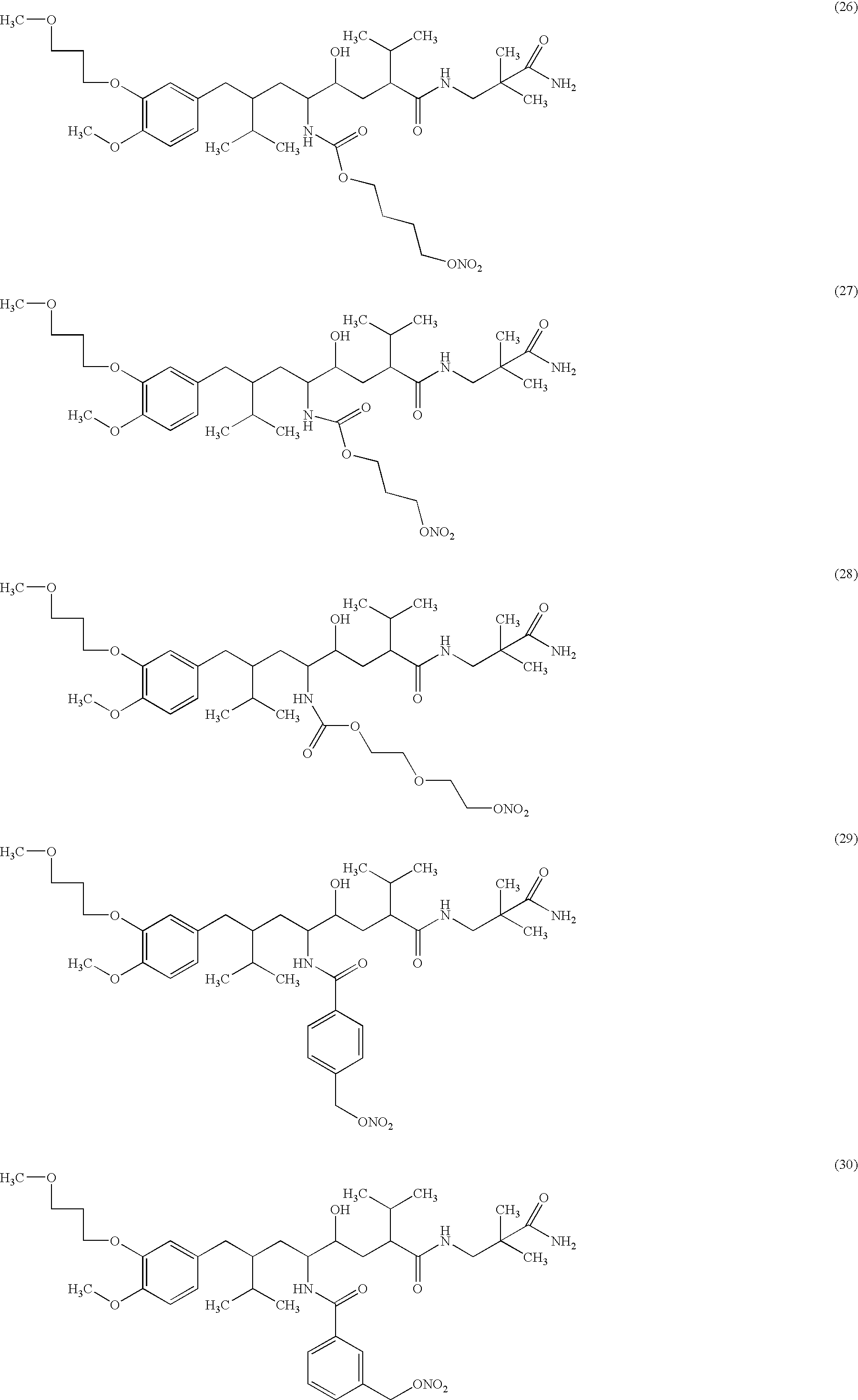

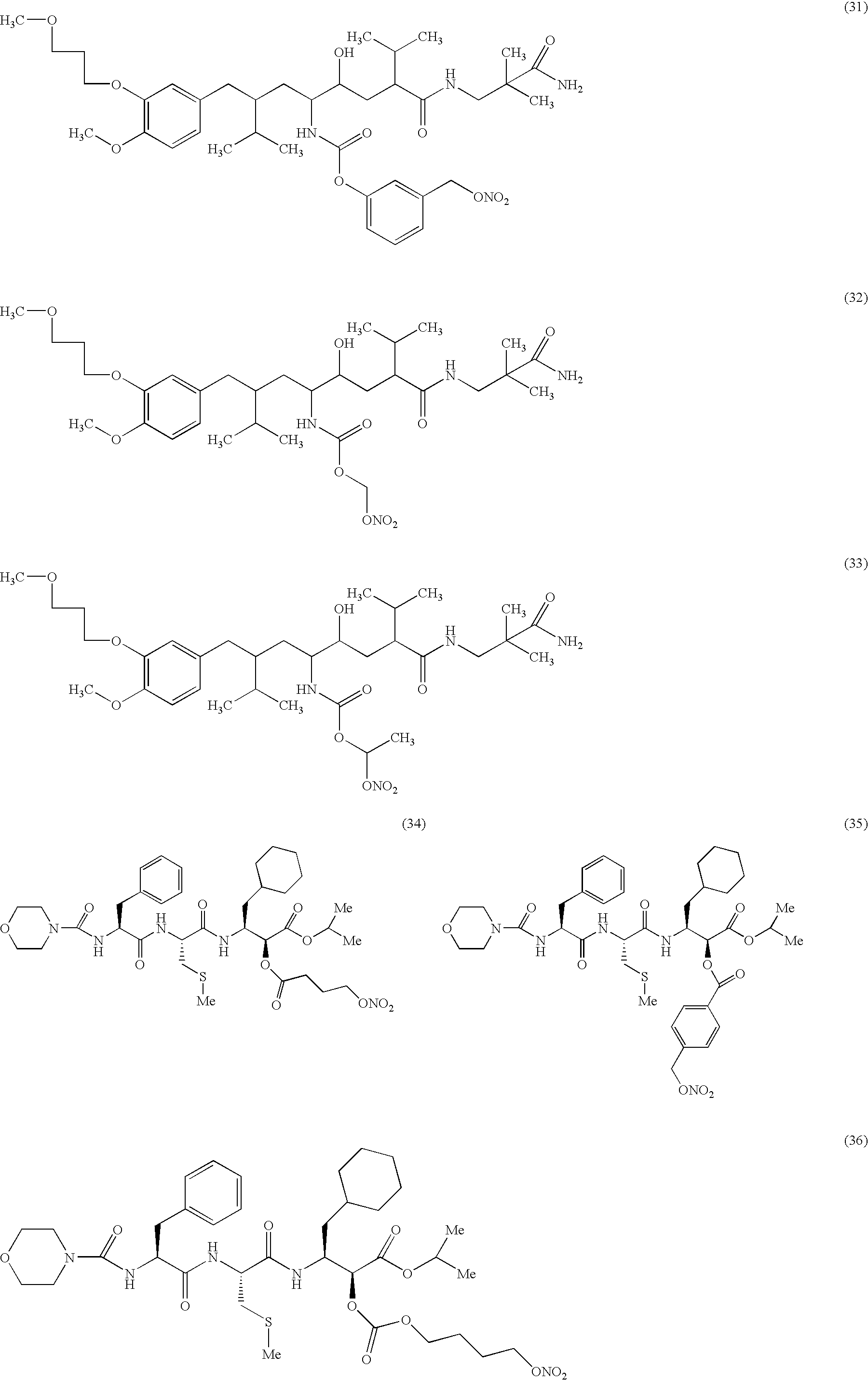

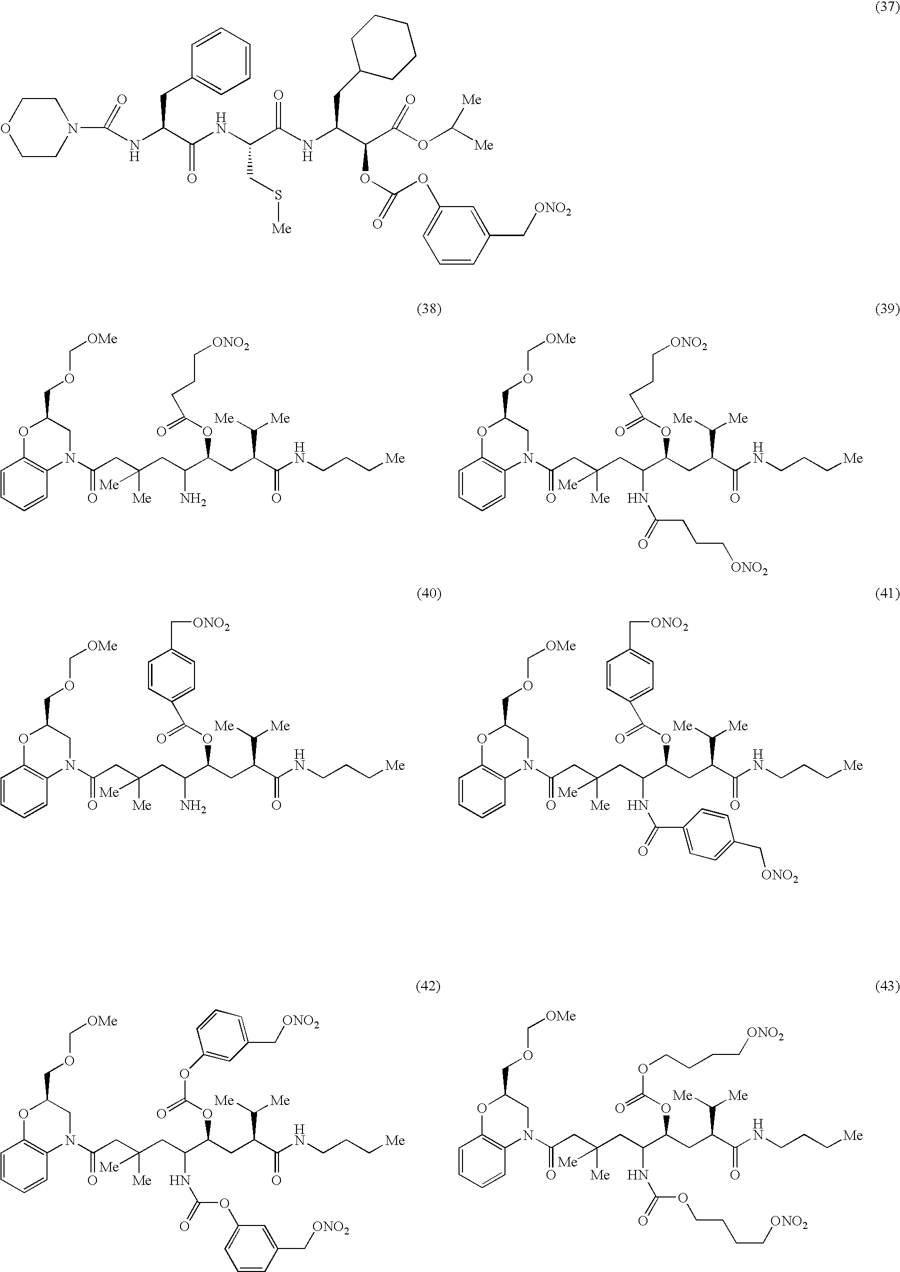

4. A compound of general formula (I) or a pharmaceutically acceptable salt or stereoisomer thereof according to claim 1 wherein A is the following formula:

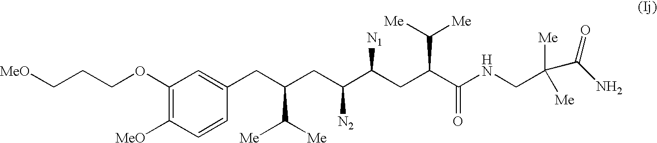

5. A compound of general formula (I) according to claim 1 for use as a medicament.

6. Use of a compound according to claim 1 for preparing a drug having anti-inflammatory, antithrombotic and antiplatelet activity.

7. Use of a compound according to claim 1, for preparing a drug that can be employed in the treatment or prophylaxis of cardiovascular, renal and chronic liver diseases, inflammatory processes and metabolic syndrome.

8. Use of a compound according to claim 7, for preparing a drug that can be employed in the treatment or prophylaxis of congestive heart failure, coronary diseases, cardiac insufficiency, left ventricular dysfunction and hypertrophy, cardiac fibrosis, myocardial ischemia, stroke, atherosclerosis, restenosis post angioplasty, renal insufficiency, renal ischemia, renal failure, renal fibrosis, glomerulonephritis, renal colic, ocular and pulmonary hypertension, glaucoma, hypertension, diabetic complications such as nephropathy, vasculopathy and neuropathy, peripheral vascular diseases, liver fibrosis, portal hypertension, metabolic syndrome, erectile dysfunction, complications after vascular or cardiac surgery, complications of treatment with immunosuppressive agents after organ transplantation, hyperaldosteronism, lung fibrosis, scleroderma, anxiety, cognitive disorders.

9. A pharmaceutical composition comprising a pharmaceutically acceptable carrier and a pharmaceutically effective amount of a compound of general formula (I) or a salt or stereoisomer thereof according to claim 1.

10. A pharmaceutical composition according to claim 9 in a suitable form for the oral, parenteral, rectal, topic and transdermic administration, by inhalation spray or aerosol or iontophoresis devices.

11. Liquid or solid pharmaceutical composition for oral, parenteral, rectal, topic and transdermic administration or inhalation in the form of tablets, capsules and pills eventually with enteric coating, powders, granules, gels, emulsions, solutions, suspensions, syrups, elixir, injectable forms, suppositories, in transdermal patches or liposomes, containing a compound of formula (I) or a salt or stereoisomer thereof according to claim 1 and a pharmaceutically acceptable carrier.

12. A pharmaceutical composition comprising a compound of general formula (I) according to claim 1, at least a compound used to treat cardiovascular disease and a pharmaceutically acceptable carrier.

13. Pharmaceutical composition according to claim 12 wherein the compound used to treat cardiovascular disease is selected from the group consisting of: aldosterone antagonists, angiotensin II receptor blockers, ACE inhibitors, HMGCoA reductase inhibitors, beta-adrenergic blockers, alpha-adrenergic antagonists, sympatholitics, calcium channel blockers, endothelin antagonists, neutral endopeptidase inhibitors, potassium activators, diuretics, vasodilators, antithrombotics such as aspirin or nitrosated compounds thereof.

14. A pharmaceutical kit comprising a compound of general formula (I) as defined in claim 1, a compound used to treat cardiovascular disease as combined preparation for simultaneous, separated, sequential use for the treatment of cardiovascular disease.

15. A pharmaceutical kit according to claim 14 wherein the compound used to treat cardiovascular disease is selected from the group consisting of: aldosterone antagonists, angiotensin II receptor blockers, ACE inhibitors, HMGCoA reductase inhibitors, beta-adrenergic blockers, alpha-adrenergic antagonists, sympatholitics, calcium channel blockers, endothelin antagonists, neutral endopeptidase inhibitors, potassium activators, diuretics, vasodilators, antithrombotics such as aspirin or nitrosated compounds thereof.
